(19) United States
(12) Patent Application Publication
(10) Pub. No.: US 2017/0007700 A1
(43) Pub. Date: Jan. 12, 2017

(54) COMPOSITIONS AND METHODS FOR BORON NEUTRON CAPTURE THERAPY

(71) Applicant: THE CURATORS OF THE UNIVERSITY OF MISSOURI, Columbia, MO (US)

(72) Inventors: Satish S. Jalisatgi, Columbia, MO (US); Marion Frederick Hawthorne, Columbia, MO (US)

(73) Assignee: The Curators of the University of Missouri, Columbia, MO (US)

(21) Appl. No.: 15/120,429

(22) PCT Filed: Feb. 23, 2015
(86) PCT No.: PCT/US15/17059

§ 371 Date: Aug. 19, 2016

Related U.S. Application Data
(60) Provisional application No. 61/943,007, filed on Feb. 21, 2014.

(51) Int. Cl. A61K 41/00; A61N 5/10; C07F 5/05; A61K 9/127; A61K 31/69

(52) U.S. Cl. CPC A61K 41/0095; A61K 9/127; A61K 31/69; C07F 5/05; A61N 5/10; A61N 2005/109

(57) ABSTRACT

Provided herein are compositions and methods for boron neutron capture therapy. In particular, the disclosure relates to therapy using liposomal Na₃[1-(2′-B₁₀H₉)-2-NH₃B₁₀H₈] and K[nido-7-CH₃(CH₂)15-7,8-C₂B₉H₁₁] sources. A composition for boron neutron capture therapy, comprising a liposome having an aqueous core and an encapsulating bilayer, wherein the aqueous core comprises Na₃[1-(2′-B₁₀H₉)-2-NH₃B₁₀H₈] (TAC) and the encapsulating bilayer comprises K[nido-7-CH₃(CH₂)15-7,8-C₂B₉H₁₁] (MAC) is also provided.

### CROSS-REFERENCE

This application claims the benefit of U.S. provisional application Ser. No. 61/943,007 filed Feb. 21, 2014, and entitled “Compositions and Methods for Boron Neutron Capture Therapy,” the entire disclosure of which is incorporated herein by reference.

### TECHNICAL FIELD

The disclosure generally relates to compositions and methods for boron neutron capture therapy. In particular, the disclosure relates to therapy using liposomes comprising Na₃[1-(2′-B₁₀H₉)-2-NH₃B₁₀H₈] (TAC) and K[nido-7-CH₃(CH₂)15-7,8-C₂B₉H₁₁] (MAC) as ¹⁰B sources.

### BACKGROUND

Boron neutron capture theory (BNCT) is a method for cancer therapy and, more particularly, for the treatment of malignant tumors. BNCT involves the selective accumulation of chemical agents containing the boron-10 (¹⁰B) isotope in cancer cells. Subsequent irradiation of the cancer cells with thermal neutrons results in capture of a thermal neutron by the ¹⁰B nucleus. This initiates a nuclear reaction in which decay of the excited ¹¹B nucleus produces a high linear energy transfer alpha particle and lithium nucleus. Because of the short trajectory of these particles, which approximately correlates with cell diameter, radiation damage is limited to cells containing ¹⁰B.

Historically, efforts to develop BNCT agents have been narrow. Currently, three compounds are approved for human trials; however, results have shown only limited success and limited improvement over conventional external-beam radiation therapy.

If ¹⁰B can be selectively accumulated into tumor/cancer cells, treatment of those cells can be accomplished with very few of the side effects typically associated with radiation. Therefore, there is a need for compositions and methods for selectively delivering and irradiating ¹⁰B compounds.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Briefly, therefore, one aspect of the present disclosure encompasses a composition for boron neutron capture therapy, comprising a liposome having a mean diameter ranging from 100 nanometers to 140 nanometers. The liposome has an aqueous core and an encapsulating bilayer. The aqueous core comprises Na₃[1-(2′-B₁₀H₉)-2-NH₃B₁₀H₈] (TAC) and the encapsulating bilayer comprises K[nido-7-CH₃(CH₂)15-7,8-C₂B₉H₁₁] (MAC). The total molar ratio of TAC to MAC in the liposome is from 1:1 to 1:6. The boron content per liposome may be from 1600 ppm to 1900 ppm, such as 1800 ppm. The liposome may have a mean diameter of less than 132 nanometers. The outer charge of the liposome may have a zeta potential ranging from −70 mV to −80 mV, such as −76 mV. The liposome may comprise cholesterol and 1,2-distearoyl-sn-glycero-3-phosphocholine. At least one of TAC or MAC may be enriched with ¹⁰B.

Another aspect of the present disclosure encompasses a method of inhibiting growth of a tumor in a subject in need thereof. The method comprises administering to a subject in need thereof any liposome described herein, and irradiating the tumor in the subject with neutrons. The time between initial administration of the liposome and irradiation of the tumor may be greater than 56 hours, such as greater than 96 hours. The ratio of boron in the tumor to the boron in the blood may reach greater than 5:1 prior to irradiation of the tumor, such as about 5.66:1 prior to irradiation. The tumor may have an increase in volume of at least 500% less than the increase in volume of a similar tumor in a control, untreated subject, for example, after 14 days.

Yet another aspect of the present disclosure provides a method for preparing K[nido-7-CH₃(CH₂)ₙ-7,8-C₂B₉H₁₁]. The method comprises contacting a compound comprising [B₁₀H₁₀]²⁻ with a strong acid and dialkylsulfide to give a sulfur-containing B₁₀H₁₂ intermediate contacting the sulfur-containing B₁₀H₁₂ intermediate with an alkyl alkyne to form a carborane; and contacting the carborane with a strong base to give K[nido-7-CH₃(CH₂)ₙ-7,8-C₂B₉H₁₁]. The strong acid may be CF₃SO₃H. The dialkylsulfide may be diethylsulfide. The sulfur-containing B₁₀H₁₂ intermediate may be B₁₀H₁₂(Et₂S)₂. And the alkyl alkyne may be CH₃(CH₂)₁₅CCH.

Another aspect of the disclosure provides a method for preparing an isomer of ¹⁰B-enriched Na₃[1-(2′-B₁₀H₉)-2-NH₃B₁₀H₈]. The method comprises contacting a compound comprising ¹⁰B-enriched decaborane with an oxidizing agent to give a B₂₀ dimer; and contacting the B₂₀ dimer with a strong base chosen from NaN(SiMe₃)₂, LiN(SiMe₃)₂, Li, Na, or NaH and ammonia to give the axial-equatorial isomer of ¹⁰B-enriched Na₃[1-(2′-B₁₀H₉)-2-NH₃B₁₀H₈]. The method further may further comprise c) contacting the axial-equatorial isomer of ¹⁰B-enriched Na₃[1-(2′-B₁₀H₉)-2-NH₃B₁₀H₈] with a strong base to give the axial-axial isomer of ¹⁰B-enriched Na₃[1-(2′-B₁₀H₉)-2-NH₃B₁₀H₈]. ¹⁰B-enriched decaborane may be chosen from ¹⁰B-enriched (HBu₃N)₂B₁₀H₁₀ and ¹⁰B-enriched (HEt₃N)₂B₁₀H₁₀. The yield of the axial-axial isomer of ¹⁰B-enriched Na₃[1-(2′-B₁₀—H₉)-2-NH₃B₁₀H₈] may be greater than 75%. The oxidizing agent may be FeCl₃.

Additional embodiments and features are set forth in part in the description that follows, and in part will become apparent to those skilled in the art upon examination of the specification, or may be learned by the practice of the embodiments discussed herein. A further understanding of the nature and advantages of certain embodiments may be realized by reference to the remaining portions of the specification and the drawings, which forms a part of this disclosure.

### BRIEF DESCRIPTION OF THE DRAWINGS

The disclosure will be readily understood by the following detailed description in conjunction with the accompanying drawings, wherein like reference numerals designate like structural elements. The drawings provide exemplary embodiments or aspects of the disclosure and do not limit the scope of the disclosure.

FIG. 1 shows K[nido-7-CH₃(CH₂)15-7,8-C₂B₉H₁₁] (MAC) synthesis starting from (HBu₃N)₂B₁₀H₁₀.

FIG. 2 shows Na₃[1-(2′-B₁₀H₉)-2-NH₃B₁₀H₈] (TAC) synthesis starting from (HBu₃N)₂B₁₀H₁₀ or (HEt₃N)₂B₁₀H₁₀.

FIG. 3 is an illustration of the synthesis of a fluorescent-labeled TAC analog.

FIG. 4 is an illustration of the chemical structure of the three main metabolites of MAC in mouse urine identified by matrix-assisted laser desorption imaging mass spectrometry (MALDI).

FIGS. 5A-B are graphs of the distribution of boron in mouse tissues over time following TAC/MAC liposomal injections (diamond=blood; square=liver; light circle=spleen; dark circle=kidney; triangle=tumor). On the data points are means and error bars depict ±SD. Sample size for each time point varied but was never less than n=4.

FIGS. 6A-C are graphs of tumor growth normalized with respect to average volume at day 0 (set as the time of irradiation): control group; □, BNCT group.

FIG. 7 shows Kaplan-Meier time-to-event curves indicating time to reach a 500-mm³ tumor volume (solid black line, control group; solid gray line, neutron only group; dashed line, BNCT group). The y-axis indicates fraction of mice having not yet developed a tumor greater than 500 mm³. Time 0 indicates the day of irradiation for the BNCT and neutron-only mice; all mice were implanted with tumor cells on the same day. The median time represents the least amount of time (in days) for 50% (0.5) of the mice to develop a tumor ≧500 mm³.

FIG. 8 depicts the particle size and polydespersity index (PDI) for liposomes formed from high-pressure extrusion.

FIG. 9 depicts the particle size and PDI for liposomes formed from the high-pressure homogenization.

FIG. 10 depicts the biodistribution of 110-nm TAC/MAC liposomes in EMT6 tumor cell line at different time points.

FIG. 11 depicts the biodistribution of 130-nm TAC/MAC liposomes at 30 hours.

FIG. 12 depicts the biodistribution in BALB/c mice bearing EMT6 tumors of TAC/MAC liposomes (100 nm, PDI of 0.164) synthesized by high-pressure homogenization.

### DETAILED DESCRIPTION

Briefly, the present disclosure provides compositions and methods for boron neutron capture therapy (BNCT). The present disclosure also provides methods of synthesizing ¹⁰B agents for boron neutron capture theory. Finally, the present disclosure further provides methods of making polyhedral boranes used in the compositions.

Without wishing to be bound by theory, BNCT takes advantage of tumors having a selective preference for boron-10 over boron-11, and of boron having a large cross-sectional radius to neutron bombardment. Taken together, boron-containing compounds turn tumor cells into selective targets for neutron bombardment. Liposomes provide greater bioavailability and stability for these boron-containing compounds.

Additional embodiments and features are set forth in part in the description that follows, and in part will become apparent to those skilled in the art upon examination of the specification, or may be learned by the practice of the embodiments discussed herein. A further understanding of the nature and advantages of certain embodiments may be realized by reference to the remaining portions of the specification the drawings, the chemical structures, and descriptions, which forms a part of this disclosure. Any description of any R-group or chemical substituent, alone or in any combination, may be used in any chemical Formula described herein, and Formulae include all conformational and stereoisomers, including diastereomers, epimers, and enantiomers. Moreover any feature of a composition disclosed herein may be used in combination with any other feature of a composition disclosed herein.

### I. Compositions for Boron Neutron Capture Therapy

In one embodiment, the disclosure provides a composition for boron neutron capture therapy. The composition comprises a liposome having a mean diameter ranging from about 90 nanometers to about 140 nanometers. The liposome has an aqueous core and an encapsulating bilayer. The aqueous core comprises Na₃[1-(2′-B₁₀H₉)-2-NH₃B₁₀H₈] (TAC) and the encapsulating bilayer comprises K[nido-7-CH₃(CH₂)15-7,8-C₂B₉H₁₁] (MAC). The total molar ratio of TAC to MAC in the liposome is from about 1:1.3 to about 1:7, such as about 1:1.6 to about 1:2, or from about 1:5.5 to about 1.6.

A liposome is a vesicle including at least one lipid bilayer. Aggregation of the hydrophilic tails of a lipid oriented inward and the polar head groups oriented outward, creating the bilayer. The bilayers are generally spherical and encapsulate an aqueous volume, which is referred to herein as the aqueous core. The bilayers may also be characterized by an internal space created between the layers of hydrophilic tails, and this space is referred to herein as the encapsulating bilayer.

The size of the liposome may impact how much boron can be incorporated in the liposome and the stability of the liposome. The size of the liposome may also impact the therapeutic properties of the liposome such as its ability to deliver boron selectively. Because the liposomes are generally spherical, they can be characterized by a mean diameter. In one embodiment, the mean diameter ranges from about 60 nanometers to about 160 nanometers. In another embodiment, the mean diameter of the liposome ranges from about 100 nanometers to about 140 nanometers. In still other embodiments, the mean diameter ranges from about 100 nanometers to about 110 nanometers, from about 105 nanometers to about 115 nanometers, from about 110 nanometers to about 120 nanometers, from about 115 nanometers to about 125 nanometers, from about 120 nanometers to about 130 nanometers, from about 125 nanometers to about 135 nanometers, from about 130 nanometers to about 140 nanometers. In still other embodiments, the mean diameter of the liposome is less than 140 nanometers, less than 139 nanometers, less than 138 nanometers, less than 137 nanometers, less than 136 nanometers, less than 135 nanometers, less than 134 nanometers, less than 133 nanometers, less than 132 nanometers, less than 131 nanometers, or less than 130 nanometers.

Without wishing to be bound by theory, the size of the liposomes may also be selected based on the method of preparation. In some embodiments, the sizes of the liposomes are in monomodal distribution. In other embodiments, the sizes of the liposomes are in bimodal distribution. In exemplary embodiments, sonication and high-pressure homogenization may provide liposomes in bimodal distribution with an average size of about 100 nm to about 140 nm. The size of the liposome may also affect the bioavailability of the particle to the tumor; i.e., the liposome's biodistribution.

A liposome may be constructed of various materials but is generally primarily constructed of fats. Liposomes are typically constructed with at least one phospholipid. Phospholipids may be chosen from phosphatidylcholine (PC), phosphatidylethanolamine (PE), phosphatidylserine (PS), phosphatidylinositol (PI); glycerophospholipids, such as phosphatid-ylglycerol, diphosphatidylglycerol (cardiolipin), phosphatidic acid (PA), sphygnomyelin; dimyris-toylphosphatidylcholine (DMPC), dipalmitoylphosphatidylcholine (DPPC), distearoylphospha-tidylcholine (DSPC), palmitoyloleoylphosphatidylcholine (POPC), dioleoylphosphatidylcholine (DOPC), dioleoylphosphatidylethanolamine (DOPE), dimyristoylphosphatidylethanolamine (DMPE), dipalmitoylphosphatidylethanolamine (DPPE), cholesterolhemisuccinate (CHEMS), cholesterol-(3-imidazol-1-ylpropyl)carbamate (CHIM), dimethyldioctadecylammonium bromide (DDAB), (1,2-dioleoxypropyl)-N,N,N-trimethylammonium salt (DOTAP), dioleoylphosphatidyl-serine (DOPS), dioleoylphosphatidylglycerol (DOPG), cholesterol sulfate (Chol-SC), 4-(2-aminoethyl)-morpholinocholesterolhemisuccinate (MoChol), histaminylcholesterolhemisuccinate (HisChol), 1,2-dipalmitoylglycerol-3-hemisuccinate (DGSucc), 1,2-distearoylglycerol-3-hemisuccinate, 1,2-dimyristoylglycerol-3-hemisuccinate, 1,2-dioleoylglycerol-3-hemisuccinate, palmitoyloleolylglycerol-3-hemisuccinate, and the like.

The liposomes may also contain fluorophores such as 4-nitro-2-oxa-1,3-diazole (NBD), lissamine rhodamine B, dansyl pyrene, or fluorescene attached to the headgroup. The phospholipid may be combined with one or more additional lipids. Additional lipids may be chosen from various sterols including cholesterol and possibly incorporating a percentage of sterols including cholesterol and possibly incorporating a percentage of sterols fluorescently labeled with either 4-nitro-2-oxa-1,3-diazole (NBD), dipyrrometheneborontrifluoride (BODYPY) or the inherently fluorescent dehydroergosterol (DHE). In one embodiment, the liposome is constructed of 1,2-distearoyl-sn-glycero-3-phosphocholine and cholesterol. The molar ratio of 1,2-distearoyl-sn-glycero-3-phosphocholine and cholesterol to cholesterol may, in different embodiments, range from 3:1 to 1:1 1,2-distearoyl-sn-glycero-3-phosphocholine to cholesterol. In another embodiment, the molar ratio of 1,2-distearoyl-sn-glycero-3-phosphocholine to cholesterol is 2:1. In still another embodiment, the molar ratio of 1,2-distearoyl-sn-glycero-3-phosphocholine to cholesterol is 1:1. Such liposomes may be prepared by methods known in the art or as prepared in Example 2.

One or more polyhedral boranes are incorporated into the liposome to provide the ¹⁰B source. Generally, a lipophilic polyhedral borane is encapsulated by the liposome's core, while a hydrophilic polyhedral borane is encapsulated in the encapsulating bilayer. In one embodiment, the aqueous core comprises Na₃[1-(2′-B₁₀H₉)-2-NH₃B₁₀H₈] (TAC) and the encapsulating bilayer comprises K[nido-7-CH₃(CH₂)15-7,8-C₂B₉H₁₁] (MAC). In still another embodiment, the aqueous core comprises a fluorescently labeled Na₃[1-(2′-B₁₀H₉)-2-NH₃B₁₀H₈] (TAC) and the encapsulating bilayer comprises K[nido-7-CH₃(CH₂)15-7,8-C₂B₉H₁₁](MAC).

The molar ratio of TAC to MAC in the liposome has surprisingly been found to be important to the effectiveness of BNCT. Generally, the molar ratio of TAC to MAC is about 1:1 to 1:6. In one embodiment, the molar ratio of TAC to MAC is about 1:5 to 1:7. In other embodiments, the molar ratio of TAC to MAC is about 1:5.5 to 1:6. In particular embodiments, the molar ratio of TAC to MAC is about 1:5.5, about 1:5.6, about 1:5.7, about 1:5.8, about 1:5.9, or about 1:6. In another embodiment, the molar ratio of TAC to MAC is about 1:1.6 to about 1:2. In an exemplary embodiment, the molar ratio of TAC to MAC is about 1:3 to about 1:6. In another exemplary embodiment, the molar ratio of TAC to MAC is about 1:1.3 to about 1:2.

The molar ratio of TAC to MAC in the liposome may also be selected based on the method of preparation, such as sonication, high-pressure homogenization, and high-pressure extrusion. By way of example, with sonication and homogenization, a TAC to MAC molar ratio from between about 1:3 to about 1:6, may be obtained. Using high-pressure extrusion, a TAC to MAC molar ratio of about 1:1.3 to about 1:2 may be obtained.

The total boron content of the liposome can and will vary. In some embodiments, the total boron content ranges from about 1200 ppm to about 2000 ppm. In another embodiment, the total boron content ranges from about 1600 ppm and about 1900 ppm. In still another embodiment, the total boron content of the liposome is about 1500 ppm, about 1600 ppm, about 1700 ppm, about 1800 ppm, about 1900 ppm, or about 2000 ppm.

For example, for 200 μl injected dose of liposome (TAC/MAC=1:5.5) at 1200 ppm boron the TAC is 3.1949×10⁻⁷ mol (1.0212×10⁻⁴ g) and MAC is 1.7570×10⁻⁶ mol (6.9741×10⁻⁴ g); for 2000 ppm TAC is 5.3241×10⁻⁷ mol (1.7003×10⁻⁴ g) and MAC is 2.9283×10⁻⁶ mol (1.1623×10⁻³ g). For 1:6 TAC/MAC molar ratio at 1200 ppm boron, the TAC is 3.002×10⁻⁷ mol (9.5814×10⁻⁵ g) and MAC is 1.8001×10⁻⁶ mol (7.1454×10⁻⁴ g); for 2000 ppm TAC is 5.004×10⁻⁷ mol (1.5969×10⁻⁴ g) and MAC is 3.0002×10⁻⁶ mol (1.1909×10⁻³ g).

“Isotopic enrichment” or “isotopically-enriched” refers to a substance where the relative abundance of the isotopes of a given element are altered, thus producing a form of the element that has been enriched in one particular isotope and deleted in its other isotopic forms. Elemental boron has naturally occurring abundances of about 19.9% ¹⁰B and about 80.1% ¹¹B, thus providing a ¹⁰B/¹¹B ratio of about 1:4.

In some embodiments, the composition may be isotopically enriched to contain a greater amount of ¹⁰B than naturally occurs (i.e., “¹⁰B-enrichment” or “¹⁰B-enriched”). In various embodiments, the percentage of ¹⁰B may be greater than 19.9%, 20%, 25%, 30%, 35%, 40%, 45%, 50%, 55%, 60%, 65%, 70%, 80%, 85%, 90%, 95%, 96%, 97%, 98%, 99%, 99.9%, or 99.99%, or any values in between. In other embodiments, the percentage of ¹⁰B may be from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 95% to about 99% or from about 95% to about 99.9%. In some embodiments, the percentage of ¹⁰B may be from about 20% to about 30%, from about 30% to about 40%, from about 40% to about 50%, from about 50% to about 60%, from about 60% to about 70%, from about 70% to about 80%, from about 80% to about 90%, or from about 90% to about 99%. In an exemplary embodiment, the percentage of ¹⁰B may be 95% or greater.

In some aspects, the TAC and MAC containing liposomes are characterized by a zeta potential (ζ-potential) on the outer surface of the liposome. In some embodiments of the MAC and TAC liposomes, the zeta potential may be from about −35 mV to about −85 mV. In other embodiments, the zeta potential may be about −70 mV to about −80 mV. In alternate embodiments, the zeta potential of the liposome may be about −70 mV, about −71 mV, about −72 mV, about −73 mV, about −74 mV, about −75 mV, about −76 mV, about −77 mV, about −78 mV, about −79 mV, or about −80 mV. In some embodiments, the zeta potential is negative, as it is thought to prevent liposomes clearing from the reticular endothelial system. A MAC-only liposome has a zeta potential of about −40.7 mV, while a TAC only liposome has a zeta potential of +12.8 mV.

In some embodiments, the compositions show increased selectivity for tumors over blood or other tissue. The increased selectivity causes accumulation, leading to greater concentrations of the boron agent located within the tumor. Increased selectivity for tumors also allows for increased boron concentrations at the tumor with a lower total administration.

### II. Method of Administration

The disclosure also provides a method for treating tumors. The method comprises a) administering to a subject in need thereof any liposome described herein, for example liposomes described above at Section (I); and b) irradiating the tumor in the subject with neutrons.

The liposomes may be administered to the subject by a variety of routes. For example, the liposomes may be administered orally or parenterally. In an embodiment, the liposomes are administered by injection. In one embodiment, liposomes are injected more than one time to achieve higher boron concentrations in the subject. In such embodiments, a first injection is followed by at least one additional injection. A second injection may be about 18 hours, about 20 hours, about 22 hours, about 24 hours, about 26 hours, about 28 hours, about 30 hours, or about 32 hours after the initial injection. In still other embodiments, additional injections (i.e. third or fourth injections) are given after about 24 hours, about 36 hours, about 48 hours, or about 72 hours after the initial injection.

Without being bound to any theory, total ¹⁰B concentrations prior to irradiation may reach high levels due to the selectivity of the liposomal compositions and also due to the method of injection. Generally, the amount of ¹⁰B per gram tumor may be between about 35 μg and about 66 μg. In one embodiment, the amount of ¹⁰B per gram tumor is greater than 35 μg, greater than 36 μg, greater than 37 μg, greater than 38 μg, greater than 39 μg, greater than 40 μg, greater than 41 μg, greater than 42 μg, greater than 43 μg, greater than 44 μg, greater than 45 μg, greater than 46 μg, greater than 47 μg, greater than 48 μg, greater than 49 μg, greater than 50 μg, greater than 51 μg, greater than 52 μg, greater than 53 μg, greater than 54 μg, greater than 55 μg, greater than 56 μg, greater than 57 μg, greater than 58 μg, greater than 59 μg, greater than 60 μg, greater than 61 μg, greater than 62 μg, greater than 63 μg, greater than 64 μg, greater than 65 μg, or greater than 66 μg.

A dosage form of the liposomes may comprise saline and, optionally, a pharmaceutical excipient. A variety of excipients commonly used in pharmaceutical formulations may be selected on the basis of several criteria such as, for example, the desired dosage form and the release profile properties of the dosage form. Non-limiting examples of suitable excipients include an agent selected from the group comprising a binder, a filler, a non-effervescent disintegrant, an effervescent disintegrant, a preservative, a diluent, a flavoring agent, a sweetener, a lubricant, an oral dispersing agent, a coloring agent, a taste masking agent, a pH modifier, a stabilizer, a compaction agent, and combinations of any of these agents. The amount and types of excipients may be selected according to known principles of pharmaceutical science.

In one embodiment, the excipient may be a binder, which holds the pharmaceutical composition together until administration. Suitable binders include starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, peptides, and combinations thereof.

In another embodiment, the excipient may be a filler, which adds bulk to the pharmaceutical composition for easier handling and more accurate dosing. Suitable fillers include carbohydrates, inorganic compounds, and polyvinylpyrrolidone. By way of non-limiting example, the filler may be calcium sulfate, e.g. both di- and tri-basic calcium sulfate; starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may be a non-effervescent disintegrant, which allows the pharmaceutical composition to more easily dissolve after administration without evolving gas. Suitable examples of non-effervescent disintegrants include starches (such as corn starch, potato starch, and the like), pregelatinized and modified starches thereof, sweeteners, clays (such as bentonite), microcrystalline cellulose, alginates, sodium starch glycolate, and gums (such as agar, guar, locust bean, karaya, pecitin, and tragacanth).

In another embodiment, the excipient may be an effervescent disintegrant, which allows the pharmaceutical composition to more easily dissolve during administration while evolving gas. By way of non-limiting example, suitable effervescent disintegrants include sodium bicarbonate in combination with citric acid, and sodium bicarbonate in combination with tartaric acid.

The excipient may comprise a preservative, which increases the stability and storage lifetime of the pharmaceutical composition, particularly delaying unwanted degradation of the active ingredient. Suitable examples of preservatives include antioxidants (such as alpha-tocopherol or ascorbate) and antimicrobials (such as parabens, chlorobutanol or phenol). In other embodiments, an antioxidant such as butylated hydroxytoluene (BHT) or butylated hydroxyanisole (BHA) may be used.

In another embodiment, the excipient may include a diluent, which diminishes the relative concentrations of other components within the pharmaceutical composition. Diluents suitable for use include pharmaceutically acceptable saccharides such as sucrose, dextrose, lactose, microcrystalline cellulose, fructose, xylitol, and sorbitol; polyhydric alcohols; starches; pre-manufactured direct compression diluents; and mixtures of any of the foregoing.

The excipient may include flavoring agents. Flavoring agents may be selected from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oils (such as lemon oil, orange oil, grape and grapefruit oil), and fruit essences (such as apple, peach, pear, strawberry, raspberry, cherry, plum, pineapple, and apricot).

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; stevia-derived sweeteners; chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

The excipient may comprise a surfactant, which alters the solubility parameters of the other components within the pharmaceutical composition. In various embodiments, the surfactant may be an alkylaryl polyether alcohol, such as Triton™ X-100, Surfonic™ N-100 (nonoxaynol-10), or Witconol™ NP-100; or a poloxamer, such as Pluronic™, Synperonic™, or Kolliphor™. Other suitable examples of surfactants include, for example, 2-acrylamido-2-methylpropane sulfonic acid, alkyl polyglycoside, ammonium perfluorononanoate, benzalkonium chloride (BAC), benzethonium chloride (BZT), 5-bromo-5-nitro-1,3-dioxane, cetyl trimethylammonium bromide (CTAB, hexadecyltrimehtylammonium bromide, cetyl trimethylammonium chloride), cetylpridinium chloride (CPC), cyclohexyl-1-hexyl-maltopyranoside, decylmaltopyranoside, decyl polyglucose, dimethyidioctadecylammonium chloride, dioctadecyldimethylammonium bromide (DODAB), dipalmitoylphosphatidylcholine, lauryldimethylamine oxide, dodecylmaltopyranoside, magnesium laureth sulfate polyethoxylated tallow amine (POEA), octenidine dihydrochloride, octylphenoxypolyethoxyethanol (Igepal™ CA-630), octylthioglucopyranoside (OTG), ox gall, sodium nonanoyloxybenzensulfonate, sorbitan monolaurate, surfactin, and thonozonium bromide.

In another embodiment, the excipient may be a lubricant, which allows easier removal of the pharmaceutical composition from molds during manufacture and may aid administration of the pharmaceutical composition. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer, which aids dispersion of the components of the pharmaceutical composition within the subject after administration. Suitable dispersants may include starch, alginic acid, polyvinylpyrrolidones, guar gum, kaolin, bentonite, purified wood cellulose, sodium starch glycolate, isoamorphous silicate, and microcrystalline cellulose.

Depending upon the embodiment, it may be desirable to provide a coloring agent, which aids visualization and identification of the pharmaceutical composition. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in the present invention depending on the embodiment.

The excipient may include a taste-masking agent. Taste-masking materials include cellulose hydroxypropyl ethers (HPC); low-substituted cellulose hydroxypropyl ethers (L-HPC); cellulose hydroxypropyl methyl ethers (HPMC); methylcellulose polymers and mixtures thereof; polyvinyl alcohol (PVA); hydroxyethylcelluloses; carboxymethylcelluloses and salts thereof; polyvinyl alcohol and polyethylene glycol co-polymers; monoglycerides or triglycerides; polyethylene glycols; acrylic polymers; mixtures of acrylic polymers with cellulose ethers; cellulose acetate phthalate; and combinations thereof.

In various embodiments, the excipient may include a pH modifier, which may alter the solubility profile and bioavailability parameters of components within the pharmaceutical composition. In certain embodiments, the pH modifier may include sodium carbonate or sodium bicarbonate.

The weight fraction of the excipient or combination of excipients in the pharmaceutical composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the pharmaceutical composition.

The pharmaceutical compositions detailed herein may be manufactured in one or several dosage forms. Suitable dosage forms include tablets, including suspension tablets, chewable tablets, effervescent tablets or caplets; pills; powders such as a sterile packaged powder, a dispensable powder, and an effervescent powder; capsules including both soft or hard gelatin capsules such as HPMC capsules; lozenges; a sachet; a sprinkle; a reconstitutable powder or shake; a troche; pellets such as sublingual or buccal pellets; granules; liquids for oral or parenteral administration; suspensions; emulsions; semisolids; or gels. Other suitable dosage forms include transdermal systems or patches. The transdermal system may be a matrix system, a reservoir system, or a system without rate-controlling membranes.

The dosage forms may be manufactured using conventional pharmacological techniques. Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., prilling, spray drying, pan coating, melt granulation, granulation, wurster coating, tangential coating, top spraying, extruding, coacervation and the like.

The amount of active ingredient that is administered to a subject can and will vary depending upon a variety of factors such as the age and overall health of the subject, and the particular mode of administration. Those skilled in the art will appreciate that dosages may also be determined with guidance from Goodman & Goldman's The Pharmacological Basis of Therapeutics, Tenth Edition (2001), Appendix II, pp. 475-493, and the Physicians' Desk Reference (PDR).

Suitable subjects include, without limit, humans, as well as companion animals such as cats, dogs, rodents, and horses; research animals such as rabbits, sheep, pigs, dogs, primates, mice, rats and other rodents; agricultural animals such as cows, cattle, pigs, goats, sheep, horses, deer, chickens and other fowl; zoo animals; and primates such as chimpanzees, monkeys, and gorillas. In one embodiment, the subject may be a human.

One aspect of the present disclosure provides an increased administration window. As used herein, the administration window is the amount of time from an initial administration of the liposomal boron agents to the time of irradiation. In one embodiment, the administration window ranges from about 50 hours to about 120 hours. In still other embodiments, the administration window is greater than about 40 hours, greater than about 48 hours, greater than about 56 hours, greater than about 64 hours, greater than about 72 hours, greater than about 80 hours, greater than about 82 hours, greater than about 96 hours, greater than about 100 hours, greater than about 110 hours, or greater than about 120 hours. In some embodiments, the administration window is about 54 hours. In exemplary embodiment, the administration window is about 96 hours.

Irradiation can be conducted by any neutron source capable of providing neutrons such that a ¹⁰B nucleus can capture the neutron to cause the nuclear reaction that is the basis of boron neutron capture therapy. In general, neutrons have energies of about 25 keV, about 50 keV, about 75 keV, about 100 keV, about 125 keV, about 150 keV or higher. In one embodiment, the neutron flux is greater than 10¹⁰ neutrons cm³/sec, greater than 10¹¹ neutrons cm³/sec, or greater than 10¹² neutrons cm³/sec. In one embodiment, the total fluence is from about 1.6×10¹⁰ neutrons/cm² to about from about 1.6×10¹² neutrons/cm². In one embodiment, the total fluence is about 1.6×10¹² neutrons/cm². In one embodiment, the total fluence is about 1.6×10¹¹ neutrons/cm².

The sample is positioned so that the target cells are positioned in the path of the neutron beam. Without wishing to be bound by theory, upon contact with the tissue of the subject, the neutron beam diminishes to energy levels enable capture by ¹⁰B. In one embodiment, the neutron beam is a composite single-crystal filtered thermal neutron beam. See, Brockman et al. “Spectral Performance of a Composite Single-Crystal Filtered Thermal Neutron Beam for BNCT Research at the University of Missouri,” Applied Radiation and Isotopes, 67 (2009) S222-S225, which is hereby incorporated by reference in its entirety.

Throughout administration, irradiation may be provided in a pulsed or continuous manner. In one embodiment, a single treatment of irradiation is about 20 minutes, about 30 minutes, about 40 minutes, about 50 minutes, about 1 hour, about 1 hour 30 minutes or about 2 hours.

In some embodiments, the method results in an increase in volume of a tumor that is at least 500% less than the increase in volume of a similar tumor in a control, untreated subject. In still another embodiment, the method results in an increase in volume of at least 500% less than the increase in volume of a similar tumor in a control, untreated subject after 14 days.

### III. Synthesis of MAC

In still another embodiment, the disclosure provides a method for preparing K[nido-7-CH₃(CH₂)ₙ-7,8-C₂B₉H₁₁]. The method comprises a) contacting a compound comprising [B₁₀H₁₀]²⁻ with a strong acid and dialkylsulfide or tetrahydrothiophene to give a sulfur-containing B₁₀H₁₂ intermediate; b) contacting the sulfur-containing B₁₀H₁₂ intermediate with an alkyl alkyne to form a carborane; and c) contacting the carborane with a strong base to give K[nido-7-CH₃(CH₂)ₙ-7,8-C₂B₉H₁₁].

The method comprises contacting a compound comprising [B₁₀H₁₀]²⁻ with a strong acid and a dialkylsulfide. Because [B₁₀H₁₀]²⁻ has a negative two charge, generally the charge is balanced with one or more cations with a total of a plus two charge. In one embodiment, the compound comprising [B₁₀H₁₀]²⁻ is (HBu₃N)₂B₁₀H₁₀ or (Bu₄N)₂(B₁₀H₁₀) or (NH₄)₂(B₁₀H₁₀).

The compound comprising [B₁₀H₁₀]²⁻ is contacted with a proton donor. In some embodiments, the compound comprising [B₁₀H₁₀]²⁻ is contacted with a strong acid. Examples of strong acids may include hydroiodic acid, hydrobromic acid, hydrochloric acid, perchloric acid, sulfuric acid, p-toluenesulfonic acid, methanesulfonic acid, fluorosulfuric acid, and triflic acid. In one embodiment, the compound comprising [B₁₀H₁₀]²⁻ is contacted with triflic acid or hydrochloric acid. In one embodiment, the compound comprising [B₁₀H₁₀]²⁻ is contacted with triflic acid.

The compound comprising [B₁₀H₁₀]²⁻ is also contacted with a dialkylsulfide. A dialkylsulfide is a sulfur-containing compound which is substituted by two alkyl groups, which may be the same or different. Without limitation, alkyl groups may be chosen from methyl, ethyl, propyl, isopropyl, butyl, sec-butyl, t-butyl, pentyl, and hexyl. In one embodiment, the dialkylsulfide is diethylsulfide.

The amount of the compound comprising [B₁₀H₁₀]²⁻ to proton donor can and will vary. In one embodiment, the compound comprising [B₁₀H₁₀]²⁻ to proton donor ranges from about 1:1 to about 3:1. In other embodiments, the amount of the compound comprising [B₁₀H₁₀]²⁻ to proton donor is about 1:1, about 1.5:1, about 2:1, about 2.5:1, or about 3:1. In one embodiment, the amount of the compound comprising [B₁₀H₁₀]²⁻ to proton donor is about 2.1.

The amount of the compound comprising [B₁₀H₁₀]²⁻ to dialkylsulfide can and will vary. In some embodiments, the amount of the compound comprising [B₁₀H₁₀]²⁻ to dialkylsulfide is about 1:1 to about 1:20. In some embodiments, the amount of the compound comprising [B₁₀H₁₀]²⁻ to dialkylsulfide is about 1:1, about 1:5, about 1:10, about 1:15, or about 1:20. In one embodiment, the amount of the compound comprising [B₁₀H₁₀]²⁻ to dialkylsulfide is about 1:5.

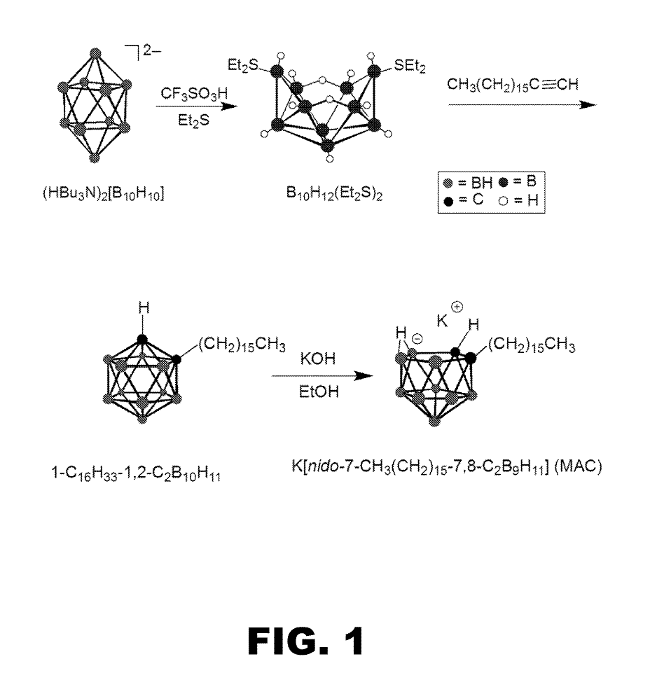

Step (a) results in a sulfur-containing B₁₀H₁₂ intermediate. As will be understood in the art, the sulfur moiety of the sulfur-containing B₁₀H₁₂ intermediate will depend on the dialkylsulfide used. In one embodiment, where the dialkylsulfide is diethylsulfide, the sulfur-containing B₁₀H₁₂ intermediate is B₁₀H₁₂ (Et₂S)₂, which is shown in FIG. 1.

The sulfur-containing B₁₀H₁₂ intermediate is then contacted with an alkyl alkyne. The sulfur-containing B₁₀H₁₂ intermediate can be used in step (b) of the reaction either as a crude product which is not further isolated from step (a), or it can be purified prior to use in step (b). The alkyl alkyne is generally a terminal alkyne, substituted at one end with an alkyl group. The alkyl group may be any alkyl group but may be selected from C₁₀ to C₂₀ alkyl groups. In one embodiment, the alkyl alkyne is CH₃(CH₂)₁₅CCH. The reaction between the sulfur containing intermediate and the alkyl alkyne gives a carborane.

According to one embodiment, the carborane may then be contacted with a proton acceptor to form K[nido-7-CH₃(CH₂)ₙ-7,8-C₂B₉H₁₁]. The carborane may be purified or contacted in the next step without further purification. In one embodiment, the carborane is not purified, but rather the crude reaction mix is used without purification. In one aspect, the proton acceptor is a strong base. Strong bases may be chosen from hydroxides such as lithium hydroxide, potassium hydroxide, sodium hydroxide, and calcium hydroxide. In one embodiment, the strong base is a mixture of potassium hydroxide and sodium hydroxide added to the carborane in excess.

The overall yield of the multi-step reaction to obtain K[nido-7-CH₃(CH₂)ₙ-7,8-C₂B₉H₁₁] can and will vary. In some embodiments, the yield will vary from about 60% to about 90%. In still another embodiment, the yield of K[nido-7-CH₃(CH₂)ₙ-7,8-C₂B₉H₁₁] is about 60%, about 65%, about 70%, about 75%, about 80%, about 85%, or about 90%.

### IV. Synthesis of TAC

In still another aspect of the present disclosure, the disclosure provides a method for preparing an isomer of Na₃[1-(2′-B₁₀H₉)-2-NH₃B₁₀H₈]. The method comprises a) contacting a compound comprising [B₁₀H₁₀]²⁻ with an oxidizing agent to give a B₂₀ dimer; b) contacting the B₂₀ dimer with strong base is chosen from NaN(SiMe₃)₂, LiN(SiMe₃)₂, Li, Na, or NaH and ammonia to give the axial-equatorial (ae) isomer of Na₃[1-(2′-B₁₀H₉)-2-NH₃B₁₀H₈]. Optionally, the method further comprises c) contacting the ae isomer of Na₃[1-(2′-B₁₀H₉)-2-NH₃B₁₀H₈] with an acid to give the axial-axial (a²) isomer of Na₃[1-(2′-B₁₀H₉)-2-NH₃B₁₀H₈].

In particular aspects of the present disclosure, the disclosure provides a method for preparing an isomer of B¹⁰-enriched Na₃[1-(2′-B₁₀H₉)-2-NH₃B₁₀H₈]. The method comprises a) contacting a compound comprising B¹⁰-enriched decaborane with an oxidizing agent to give a B₂₀ dimer; b) contacting the B₂₀ dimer with strong base is chosen from NaN(SiMe₃)₂, LiN(SiMe₃)₂, Li, Na, or NaH and ammonia to give the axial-equatorial (ae) isomer of B¹⁰-enriched Na₃[1-(2′-B₁₀H₉)-2-NH₃B₁₀H₈]. Optionally, the method further comprises c) contacting the ae isomer of B¹⁰-enriched Na₃[1-(2′-B₁₀H₉)-2-NH₃B₁₀H₈] with an acid to give the axial-axial (a²) isomer of B¹⁰-enriched Na₃[1-(2′-B₁₀H₉)-2-NH₃B₁₀H₈].

The parent [B₂₀H₁₈]⁴⁻ skeleton of the TAC can exist as three different isomer equatorial-equatorial (e²), axial-equatorial (ae), or axial-axial (a²), where two B₁₀ cages are linked by a two-electron two-center boron-boron bond. The interconversion between these isomers is pH dependent. After the synthesis, TAC is isolated as a mixture of a² and ae in the ratio of about 80:20 to about 100:0. The conversion of ae to a² proceeds through an a² ammonio derivative of [B₂₀H₁₉]³⁻ having a bridging hydrogen between two cages. This isomer is converted to the conventional a² TAC isomer after basification. See Watson-Clark et al., “Synthesis and Structure of the Elusive [a²⁻ B₂₀H₁₉]³⁻ Anion,” Inorg. Chem. 1996, 35, 2963, which is incorporated herein by reference in its entirely. The crystal structure of the ae isomer is known, as shown in Hawthorne et al., “[Na₃[B₂₀H₁₇NH₃]: Synthesis and liposomal delivery to murine tumors,” Proc. Natl. Acad. Sci. 1994, 31, 3029, which is incorporated herein by reference in its entirely. It has been discovered herein that the a² isomer can be crystalized as the ethylenediamine hydrochloride salt. The crystal structure shows that NH₃ on the TAC is linked to ethylenediamine through water molecule via hydrogen bonding.

The method comprises contacting a compound comprising [B₁₀H₁₀]²⁻ with an oxidizing agent. Because [B₁₀H₁₀0]² has a negative two charge, generally, the charge is balanced with one or more cations with a total of a plus two charge. In one embodiment, the compound comprising [B₁₀H₁₀]²⁻ is (HBu₃N)₂B₁₀H₁₀. In another embodiment, the compound comprising [B₁₀H₁₀]²⁻ is (HEt₃N)₂B₁₀H₁₀.

The oxidizing agent may be a compound comprising iron. In some embodiments, the iron is in a +2 or +3 oxidation state. In one embodiment, the iron source is FeCl₃. Other oxidizing agents known in the art may also be used, including MnO₄, MnO₂, or molecular oxygen ([O]).

The oxidizing agent is generally added such that the oxidant is provided in excess to the compound comprising [B₁₀H₁₀]²⁻. The amount of oxidizing agent provided can and will vary. In one embodiment, the amount of the compound comprising [B₁₀H₁₀]²⁻ to the oxidizing agent ranges from 1:1 to about 1:10. In another embodiment, the amount of the compound comprising [B₁₀H₁₀]²⁻ to the oxidizing agent ranges from about 1:1, about 1:1.5, about 1:2, about 1:3, about 1.3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. In one embodiment, the amount of the compound comprising [B₁₀H₁₀]²⁻ to the oxidizing agent is about 1:3.5.

Generally, the oxidizing agent is added with a proton donor in aqueous solution. Proton donors may be chosen from HCl, HBr, HI, HClO₃, HClO₄, HBrO₄, HIO₃, HIO₄, HNO₃, H₂SO₄, MeSO₃H, CF₃SO₃H, alkyl sulfonic acids, aryl sulfonic acids, and the like. In one embodiment, the proton donor is added in at least one molar equivalent. In another embodiment, the proton donor is added until a neutral pH is achieved, in still another embodiment, the proton donor is added until the pH is about 7.

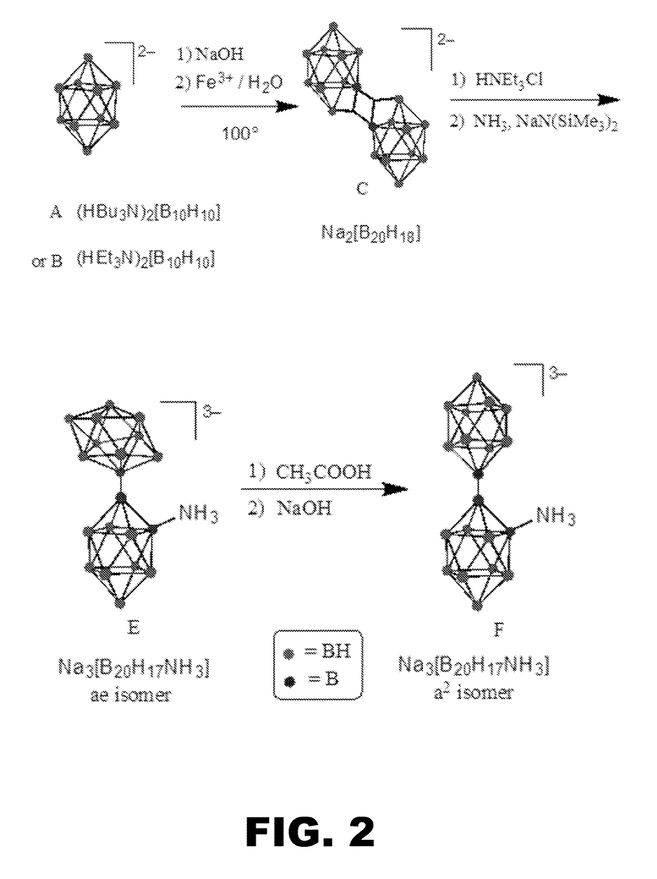

Reaction with the oxidizing agent creates a B₂₀ dimer. An exemplary B₂₀ dimer is shown in FIG. 2. In one embodiment, without further purification of the B₂₀ intermediate, an amine salt is added. The amine salt is of the general formula HN(R²)₃X wherein each R² is independently selected from hydrocarbyl and substituted hydrocarbyl and X is a leaving group. In exemplary embodiments, each R² may be independently selected from a C₁-C₆ alkyl, and X is chloride. In one embodiment, the amine salt is HNEt₃Cl.

The product formed is the contacted with a strong base in ammonia where the strong base is chosen from NaN(SiMe₃)₂, LiN(SiMe₃)₂, Li, Na, or NaH in ammonia. In general, this reaction is conducted in a closed, ventable vessel such as an autoclave. Reaction with a strong base and ammonia gives the ae isomer of Na₃[B₂₀H₁₇NH₃].

Optionally, reaction with a strong base can be conducted to give the a² isomer Na₃[B₂₀H₁₇NH₃]. Strong bases may be chosen from hydroxides such as lithium hydroxide, potassium hydroxide, sodium hydroxide, and calcium hydroxide. A figure of the a² isomer is shown at FIG. 2.

The overall yield of the multi-step reaction to obtain Na₃[1-(2′-B₁₀H₉)-2-NH₃B₁₀H₈] can and will vary. The final TAC product contains almost 15 moles of water per mole of TAC as the crystallization solvent. In some embodiments, the yield will vary from about 60% to about 90%. In still another embodiment, the yield of Na₃[1-(2′-B₁₀H₉)-2-NH₃B₁₀H₈] is about 60%, about 65%, about 70%, about 75%, about 80%, about 85%, or about 90%.

### DEFINITIONS

The compounds described herein have asymmetric centers. Compounds of the present disclosure containing an asymmetrically substituted atom may be isolated in optically active or racemic form. All chiral, diastereomeric, racemic forms and all geometric isomeric forms of a structure are intended, unless the specific stereochemistry or isomeric form is specifically indicated.

The term “acyl,” as used herein alone or as part of another group, denotes the moiety formed by removal of the hydroxy group from the group COOH of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl, or substituted hydrocarbyl.

The term “acyloxy,” as used herein alone or as part of another group, denotes an acyl group as described above bonded through an oxygen linkage (O), e.g., RC(O)O— wherein R is as defined in connection with the term “acyl.”

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “halogen” or “halo” as used herein alone or as part of another group refer to chlorine, bromine, fluorine, and iodine.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “oxygen-protecting group” as used herein denotes a group capable of protecting an oxygen atom (and hence, forming a protected hydroxyl group), wherein the protecting group may be removed, subsequent to the reaction for which protection is employed, without disturbing the remainder of the molecule. Exemplary oxygen protecting groups include ethers (e.g., allyl, triphenylmethyl (trityl or Tr), p-methoxybenzyl (PMB), p-methoxyphenyl (PMP)), acetals (e.g., methoxymethyl (MOM), β-methoxyethoxymethyl (MEM), tetrahydropyranyl (THP), ethoxy ethyl (EE), methyithiomethyl (MTM), 2-methoxy-2-propyl (MOP), 2-trimethylsilylethoxymethyl (SEM)), esters (e.g., benzoate (Bz), allyl carbonate, 2,2,2-trichloroethyl carbonate (Troc), 2-trimethylsilylethyl carbonate), silyl ethers (e.g., trimethylsilyl (TMS), triethylsilyl (TES), triisopropylsilyl (TIPS), triphenylsilyl (TPS), t-butyldimethylsilyl (TBDMS), t-butyldiphenylsilyl (TBDPS) and the like. A variety of oxygen protecting groups and the synthesis thereof may be found in “Protective Groups in Organic Synthesis” by T. W. Greene and P. G. M. Wuts, 3^{rd} ed., John Wiley & Sons, 1999.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

When introducing elements of the present disclosure or the embodiments(s) thereof, the articles “a,” “an,” “the,” and “said” are intended to mean that there are one or more of the elements. The terms “comprising,” “including,” and “having” are intended to be inclusive and mean that there may be additional elements other than the listed elements.

### EXAMPLES

The following examples are included to demonstrate preferred embodiments. It should be appreciated by those of skill in the art that the techniques disclosed in the examples represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth is to be interpreted as illustrative and not in a limiting sense.

### Example 1

### Synthesis of Compounds

Boron 10-enriched K[nido-7-CH₃(CH₂)15-7,8-C₂B₉H₁₁] (MAC) and Na₃[1-(2′-B₁₀H₉)-2-NH₃B₁₀H₈] (TAC) were prepared under inert (argon) atmospheres beginning from ¹⁰B-enriched decaborane, with the percentage of ¹⁰B being 95% or greater. FIG. 1 illustrates the synthesis of MAC starting from (HBu₃N)₂B₁₀H₁₀. FIG. 2 illustrates the synthesis of TAC starting from (HBu₃N)₂B₁₀H₁₀ or (HEt₃N)₂B₁₀H₁₀.

(HBu₃N)₂B₁₀H₁₀ was synthesized to 6,9-(Et₂S)₂—B₁₀H₁₂ as seen in the first step of FIG. 1. To an ice-bath cooled suspension of (HBu₃N)₂B₁₀H₁₀ (1.00 g, 1.66 mmol) in 10 mL of Et₂S was added dropwise CF₃SO₃H (0.73 mL, 8.30 mmol). The resulting reaction mixture was stirred at room temperature (RT) for 1.5 h. The top layer (Et₂S) was then separated and the residue was extracted with 5 mL of Et₂S. The combined diethylsulfide phases were evaporated to dryness, dried under vacuum, and used in the next step without further purification (400 mg of yellowish oil, 80%).

Alternatively, MAC may be formed from B₁₀H₁₀(NH₄)₂ and HCl, as shown in Scheme 1 below. To an oven-dried 420-mL high-pressure glass reactor, 1 g (6.49 mmol) of B₁₀H₁₀(NH₄)₂ was suspended in 20 mL (185 mmol) of distilled diethylsulfide (Et₂S). To this mixture, 1 mL of condensed HCl gas (32.65 mmol) was added to and the flask was capped. The reaction mixture was allowed to stir at room temperature for 24 hour. The ¹¹B-NMR of the mixture showed the formation of B₁₀H₁₂(Et₂S)₂. The solution was filtered under argon and the filtrate was concentrated under vacuum using nitrogen trap to give 0.520 g (1.72 mmol) (80%) of B₁₀H₁₂(Et₂S)₂.

Also was obtained about 0.800 g (expected 0.900 g) of a double salt NH₄Cl.(NH₄)₂B₁₀H₁₀ as a precipitate which did not dissolve in Et₂S. Next, 0.520 g (1.72 mmol) of B₁₀H₁₂(Et₂S)₂ was dissolved in 10 mL dry toluene and 0.430 g (1.72 mmol) of 1-octadecyne was added to it. The reaction mixture was stirred at 100° C. for 12 hours. The ¹¹B-NMR of crude product showed completion of reaction.

The organic layer was washed with 25 mL of 1M KOH solution and volatiles were removed under vacuum and the resulting solid was purified by silica-gel column chromatography using hexane as eluent to afford 0.337 g (53%) of closo-MAC. The purified closo-MAC (0.337 g, 0.915 mmol) was dissolved in 10 mL of ethanol and 0.153 g (2.745 mmol) of KOH was added. The reaction mixture was then refluxed for 12 h. The reaction mixture was concentrated via rotatory evaporation, and the resulting solid was recrystallized from toluene to obtain 0.230 g of MAC. Overall yield from B₁₀H₁₀(NH₄)₂ was 30%.

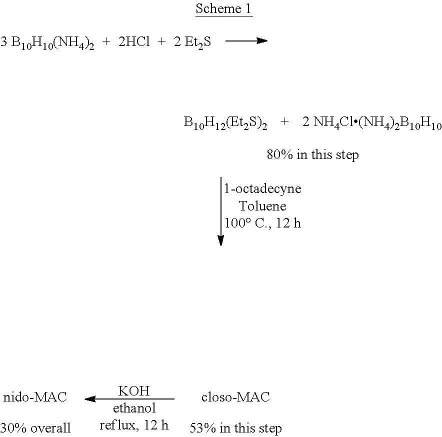

Alternatively, MAC may be synthesized using tetrahydrothiophene, as depicted in Scheme 2 below. To an oven-dried 420-mL high-pressure glass reactor, 1 g (6.49 mmol) of B₁₀H₁₀(NH₄)₂ was suspended in 20 mL (227 mmol) of distilled tetrahydrothiophene (THT). To this mixture, 1 mL of condensed HCl gas (32.65 mmol) was added and the flask was capped. The reaction mixture was allowed to stir at room temperature for 20 hours.

The ¹¹B-NMR of the reaction mixture showed the formation of B₁₀H₁₂(THT)₂. The solution was filtered under argon and the filtrate was concentrated under vacuum using nitrogen trap to give 0.551 g (1.85 mmol) (84%) of B₁₀H₁₂(THT)₂. Also obtained was about 0.900 g (expected 0.900 g) of a double salt NH₄Cl.(NH₄)₂B₁₀H₁₀ as a insoluble solid. Next, 0.551 g (1.85 mmol) of B₁₀H₁₂(THT)₂ was dissolved in 10 mL of dry toluene and 0.462 g (1.85 mmol) of 1-octadecyne was added to it. The reaction mixture was stirred at 100° C. for 12 h. The ¹¹B-NMR of the crude product showed completion of reaction.

The reaction mixture was washed with 25 mL of 1M KOH solution, and the volatiles were removed under vacuum. The resulting crude product was purified by silica-gel column chromatography using hexane as eluent to obtain 0.348 g (51%) of closo-MAC. This 0.348 g (0.944 mmol) of closo-MAC was dissolved in 10 mL of ethanol and 0.158 g (2.82 mmol) of KOH was added. The reaction mixture was refluxed for 12 hours, concentrated via rotatory evaporation. The resulting crude product was recrystallized from toluene to obtain 0.173 g of MAC. Overall yield from B₁₀H₁₀(NH₄)₂ was 22%.

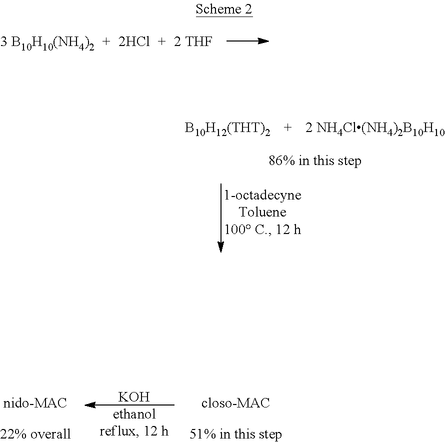

The second step of FIG. 1 illustrates the synthesis of 1-C₁₆H₃₃-1,2-C₂B₁₀H₁₁. Residue from the first step was dissolved in 5 mL of anhydrous toluene, and a solution of octadecyne (0.33 g, 1.32 mmol) in 2 mL of anhydrous toluene was added. The reaction mixture was stirred at 90° C. for 18 h. The reaction mixture was allowed to cool down to RT, evaporated to dryness, dried under vacuum, and used in the next step without further purification (490 mg of yellowish oil, 100%).

In the third step illustrated in FIG. 1 for the final synthesis of MAC, residue from the second step was dissolved in 5 mL of ethanol, and KOH (purity 85%, 0.26 g, 3.96 mmol) was added to the solution. The solution was heated to reflux for 5 hours. The reaction mixture was allowed to cool to RT and diluted with ethanol (20 mL). CO₂ was bubbled through the reaction mixture for one hour. The precipitate formed was filtered off and washed on filter with ethanol (2×5 mL). Combined ethanol fractions were evaporated to dryness and dried under vacuum. The precipitate formed was washed with cold hexane (2×5 mL) and dried under vacuum to give MAC as a white solid (0.56 g, 90%).

The starting material for TAC synthesis is either (A) (HBu₃N)₂[B₁₀H₁₀] or (B) (Het₃N)₂[B₁₀H₁₀]. Prior to use of A, the bulk material was dissolved in acetone, filtered, and dried via rotary evaporation. The solid was rinsed with diethyl ether and dried in vacuo. In the case of B, the bulk material was dissolved in a minimal amount of deionized water and filtered. The filtrate was heated to boiling with constant stirring. Absolute ethanol was added until a precipitate began to form from the boiling solution. The solution was then placed in a freezer at 0° C. and allowed to crystalize. The solid was filtered with cold (0° C.) ethanol and dried in vacuo. In some cases a second crystallization may be necessary. The second crystallization involves dissolving the solid in a minimal amount of 1:1 acetonitrile/acetone, filtering the solution, cooling to −25° C., filtering the solid, and drying in vacuo.

Step 2, using material A, 54.37 g (FW=490.90 g/mol; 0.11 mol) was suspended in 250 mL of ultrapure (18.2 MΩ·cm) water in a flask equipped with a magnetic stir bar. 13.32 g of NaOH pellets (FW=40.0 g/mol; 3 molar equivalents versus A; 0.33 mol) were added and the mixture was stirred for 1 hour. Two separate liquid layers presented upon standing. All liquid material was transferred to a separatory funnel and 250 mL of dichloromethane was added. This was shaken for 1 minute the layers were allowed to separate. The lower (organic) layer was discarded, the process was repeated two more times. The remaining aqueous layer was transferred to a large (4-L) Erlenmeyer flask equipped with a stir bar. Using material B, 250 mL of ultrapure (18.2 MΩ·cm) water was dissolved prior to step 3.

Step 3, for material A, involved adding 9.15 mL of reagent grade HCl (12.1 M; 1 molar equivalent versus A; 0.1108 mol) to the flask and stirring. The pH was measured to ensure that the pH is at least neutral. Water was added to bring the solution volume to 3 L; this was heated to boiling with continued stirring. 104.84 g of FeCl₃.6H₂O (FW=270.30 g/mol; 3.5 molar equivalents versus A; 0.39 mol) was added slowly to the flask. The flask was covered with watch glass and boiling/stirring was continued for 4 hours. After this, heat was discontinued, the material (Na₂[B₂₀H₁₈]; I was allowed to return to room temperature. Stirring was resumed and 45.74 g HNEt₃Cl (FW=137.651; 3 molar equivalents versus A; 0.33 mol) was added. Solid (HNEt₃)₂[B₂₀H₁₈] (D) precipitated from solution. D was collected via filtration and rinsed 4 times with 250 mL aliquots of ultrapure water.

The solid was allowed to reach near dryness then dissolved in minimal, boiling absolute ethanol. This was placed in a freezer (−25° C.) to promote crystallization. The solid was collected via filtration and rinsed once with 250 mL cold ethanol (−25° C.) and twice with 250 mL aliquots of diethyl ether. The solid was dried in vacuo. The yield of D was near 19.45 g (FW=438.76 g/mol; 0.04 mol; 80.1% yield). For material B, all of the above steps were performed omitting the addition of HNEt₃Cl. In a reaction starting with 112.88 g of B, yield of D was about 60.56 g (0.14 mol; 78.9% yield).

51.28 g (0.12 mol) D was placed in a 600-mL capacity stainless steel autoclave vessel. The vessel was cooled to −78° C. with an autoclave bath. Slowly 250 mL of anhydrous liquid ammonia was added, the resulting solution was kept under a stream of argon. (Some evaporation of liquid ammonia occurred.) Very slowly 75.01 g NaN(SiMe₃)₂ (FW=183.375 g/mol; 3.5 molar equivalents versus D; 0.41 mol) was added while maintaining argon stream. The vessel was sealed, warmed to 25° C. and stirred for 90 minutes. Slowly, the autoclave was vented to release gaseous ammonia. The entire interior of autoclave (including mechanical stirrer) was rinsed 3 times with 100 mL aliquots of ultrapure water. All aqueous rinses were added to a separatory funnel. 300 mL of dichloromethane was added, shaken for 1 minute, and the layers were allowed to separate. The lower (organic) layer was discarded and the process was repeated two more times. The aqueous solution was frozen to −78° C. and lyophilized (freeze dried) until no water remained (1-2 days).

200 mL of 1.17 M H₃PO₄ (FW=98.0 g/mol; 2 equivalents versus D; 0.23 mol) was added in ethanol and stirred for 2 hours at 25° C. 28.06 g NaOH (0.18 mol; 3 molar equivalents versus H₃PO₄) was added and stirred for 2 hours at 25° C. pH was tested by adding a small amount of the reaction mixture to an equal amount of water then blotting a pH test strip. The pH must be ≦10 or additional NaOH was added and the reaction continued. After the reaction is complete, slowly solid CO₂ was added to eliminate excess sodium hydroxide. More than necessary was used and stirred for 1 hour following the final addition of CO₂ to allow any foaming to subside. Solids were filtered (rinsing twice with 200 mL aliquots of ethanol), and the filtrate was dried via rotary evaporation. The residue was dissolved in 100 mL acetonitrile, filtered and rinsed 2 times with 100 mL aliquots of acetonitrile, and the filtrate was dried via rotary evaporation. Alternative, the filtrate was dried using two molar equivalents of hydrochloric acid at room temperature.

Optionally, after rotary evaporation, the material was dissolved in minimal absolute ethanol and loaded onto a plug (approximately 500 mL wet volume) of basic alumina (Brockmann activity between I and II) in ethanol. The product was eluted with 500 mL absolute ethanol then 500 mL methanol discarding the first 100 mL of eluent. Rotary evaporation was used to dry (filtering first if necessary to remove any alumina). The evaporated residue was dissolved in 500 mL of ultrapure water, frozen in a dry ice/acetone bath (−78° C.), and lyophilized to complete dryness. Yield, assuming the product has 15 moles of water of crystallization per mole of TAC (FW=589.58 g/mol), should be 49.9 g (72.4%). At this point, the product Na₃[B₂₀H₁₇(NH₃)] must be kept under dry atmosphere as it is highly hygroscopic. Weight measurements will be inaccurate if not performed under such conditions.

### Example 2

### Liposome Preparation and Analysis

The liposome nanoparticles used were artificially prepared vesicles composed of a lipid bilayer constructed from cholesterol and 1,2-distearoyl-sn-glycero-3-phosphocholine (DSPC) and contained two types of boron compounds, an amphiphilic K[nido-7-CH₃(CH₂)₁₅-7,8-C₂B₉H₁₁] (MAC) incorporated in the lipid bilayer and a hydrophilic Na₃[1-(2′-B₁₀H₉)-2-NH₃B₁₀H₈] (TAC) incorporated in the aqueous core. Liposome suspensions were created by probe sonication of dried films (300 mg) composed of DSPC (Avanti Polar Lipids), cholesterol (Aldrich), and MAC in a 1:1:0.6 molar ratio, respectively, immersed in a hypertonic wetting solution consisting of TAC (250 mM) in 6 mL water. Samples were sonicated continuously for 40 minutes in a water bath maintained at 65±0.5° C. Excess wetting solution was separated from the liposome suspension by elution from a Sephadex™ G-25 (medium) column equilibrated with isotonic phosphate-buffered lactose (5 mM phosphate/9% (wt/wt) lactose, pH 7.4) or phosphate-buffered saline (PBS) solution (10 mM phosphate/2.7 mM KCl/137 mM NaCl, pH 7.4). Liposome suspensions were diluted with the appropriate buffer to a final volume of 12 mL and filtered through two sterile 0.2-μm syringe filters into sterile serum bottles.

Smaller batch liposomes were prepared by the sonication method and larger batch liposomes were prepared by the homogenization method. The sonication method consists of suspending a dry lipid mixture in the desired wetting solution and sonicating for 15-30 minutes at about 65° C. The lipid mixture may contain a lipophilic boron derivative, and/or the wetting solution may be an aqueous borate or simply a buffer.

The lipid mixture contained equimolar mixture of distearoylphosphatidyl-choline (DSPC) and cholesterol (CH). For a 6 mL sonication, this corresponds to 202 mg DSPC and 98 mg CH. The lipids, including lipophilic boron species if used, were first dissolved in about 2 mL CHCl₃; methanol may help to produce a clear solution, otherwise the solution was filtered. Drying the solution was accomplished by either of the following two methods. (1) The solution was concentration via rotary evaporation in the culture tube at RT or below. Once the mixture was solidified, it was dried under vacuum for several hours or overnight, as traces of organic solvents may cause problems with liposome stability and reproducibility. Or (2) The solution was prefrozen in the culture tube with dry ice/acetone, and evaporated on the Speed-Vac™. The solid residue was dried under vacuum for several hours or overnight, for example on the lyophilizer.

For the incorporation of a boronated lipophile, at least enough compound to provide 10 mg of boron was used. This quantity allows for an incorporation efficiency of 70% while still providing a reasonable injected dose (5 mg/kg). If the boronated lipophile added was charged, this will limit the amount that can be usefully incorporated. Although a small amount of charge can enhance liposomal stability, too much is detrimental; so 10 mole % is used. For example, when using MAC16 (potassium hexadecyldicarbolide), the formulation DSPC/CH/MAC16 in the proportion 5:5:2 was used. Increasing the concentration of MAC16 provides a larger dose of boron but less favorable biological performance due to the reduced stability of the liposomes.

Six mL of wetting solution was used and filtered through a 0.2-μm membrane. For the preparation of “empty” liposomes having no encapsulated water-soluble boron materials, the wetting solution should was buffer, such as PBS or a hypertonic buffer to simulate the osmotic pressure of an encapsulated borane solution. The hypertonic buffer was produced by raising the NaCl concentration of PBS from 140 mM to 350 mM. For the encapsulation of water-soluble boron species, the concentration was up to a maximum osmolarity of 800 mOsM.

To ensure homogeneity, liposomes were formed (or later annealed) above the transition temperature of the lipid (57° C. for DSPC). For a small, brief sonication, a beaker of water was sufficient to control the temperature. A 150 or 250-mL beaker was filled with water and heated on a hotplate to 65±2° C. About two-thirds of the wetting solution was placed into a culture tube containing the dry lipid mixture and vortexed briefly. The remainder of the wetting solution was then added to the culture tube. The solution was then sonicated for 15 to 30 minutes until the solution became more homogenous and translucent. Once the liposomes were formed by sonication, they were separated from the wetting solution, diluted with the appropriate buffer to a final volume of 12 mL, and filtered through two sterile 0.2-μm syringe filters into sterile serum bottles. This procedure was used for the sonication of 4-6 mL samples, thereby producing 8-12 mL of liposomes.

The homogenization method continuously circulates a lipid/aqueous mixture, forcing it at high pressure (>1000 atm) through a single small orifice. The degree of homogenization (and liposome size) provided by the operating pressure is controlled by a screw valve, which adjusts the size of the orifice. The piston operates with a maximum compression ratio of about 225, thereby providing the high pressures required to produce small liposomes.

All bilayer components (phospholipid, cholesterol, lipophilic addends) were mixed thoroughly. The lipids were dissolved in CHCl₃. If necessary to produce a clear solution, about 10% of MeOH was added. In some cases, the solution was filtered through a clean fritted glass funnel. The solution was dried via rotary evaporation, spinning rapidly at RT or below, and dried thoroughly in vacuo. The dried solid was crushed to eliminate large particles.

The lipid and wetting solution were combined to create the sample that was then preheated in a water bath at 70±5° C. The homogenizer was supplied with 80-115 psi gas pressure. The sample was then placed in the sample hopper of the homogenizer and circulated for about 10 minutes. The sample pressure was raised quickly to a peak pressure of 15,000-17,000 psi, and the effluent was collected and recirculated back to the sample hopper at least two times to complete homogenization.

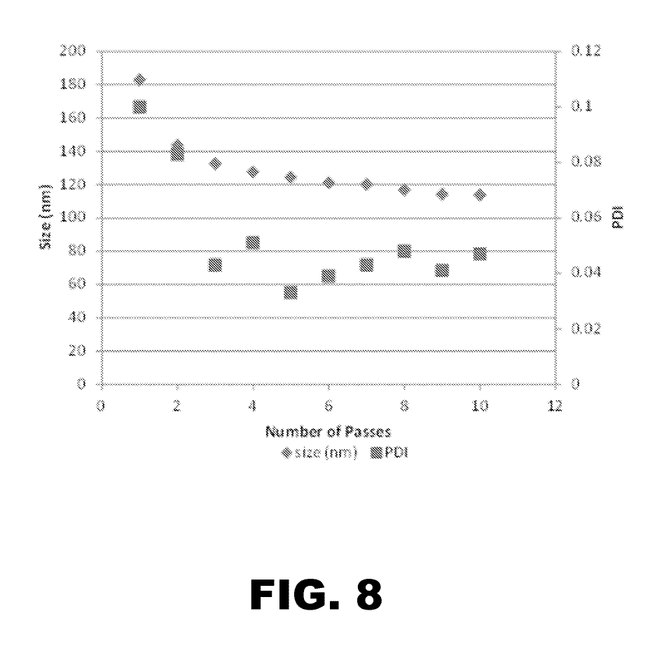

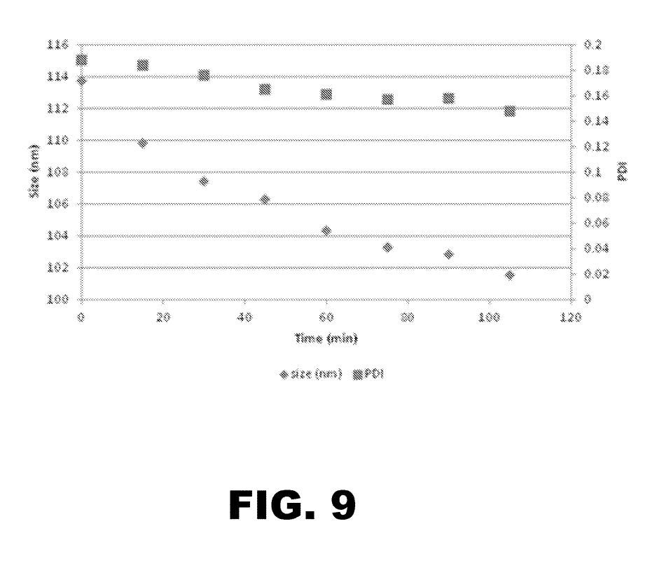

Liposomes were prepared in three different ways—sonication, high-pressure homogenization, and high-pressure extrusion. With sonication and homogenizer processes, TAC/MAC molar ratios of between 1:3 and 1:6 were obtained. With high-pressure extrusion TAC/MAC molar rations of between 1:1.3 to 1:2 were obtained. The sonication and homogenization processes each gave liposomes in bimodal distribution with an average particle size of 100 nm to 140 nm. The sonication batches were run on smaller scale and it gave considerable variation in liposome sizes from batch to batch, from 100 nm to 140 nm. The polydespersity index (PDI) was high, around 0.25. High-pressure homogenization was more consistent and gave liposomes in the 100 nm ranges with a high PDI still around 0.17. From the high-pressure extrusion, the liposome component mixture was passed through a series of filters under high pressure until the desired particle size was obtained. The PDI was always low near about 0.05, giving consistent batch-to-batch results (FIGS. 8 & 9).

A mixture of DSPC, cholesterol, and MAC in chloroform was evaporated into a film, in a similar way to homogenization. To this lipid film was added 400 mmol TAC solution in water. The mixture was stirred and transferred to the homogenizer. The homogenizer run was carried out at 70° C. for 10-15 minutes to get multilamellar liposomes. This liposome mixture was then transferred to the extruder containing a 100-nm polycarbonate filter. The extruder was kept at 65-70° C. for the whole duration. The extruder was operated at about 400 psi to about 700 psi and the process was repeated 3-4 times. A sample was analyzed for particle size. Once the particle size reached about 110 nm, the mixture was transferred to the Sephadex™ G-25 size exclusion column. The liposome fraction was separated and analyzed for any free unincorporated TAC. The boron content was measured by ICP-OES.

Typically, liposomes after purification have boron in the range of about 2400 ppm to about 3000 ppm. The liposomes were then diluted with PBS buffer to 1800 ppm. The zeta potential was similar to those liposomes made by homogenization. Small-scale liposome batches were sterilized by passing the formulation through 0.2-micron filter into a sterile bottle. The large-scale liposome batches were sterilized by autoclaving them in a steam autoclave for 10-15 minutes at 120° C.

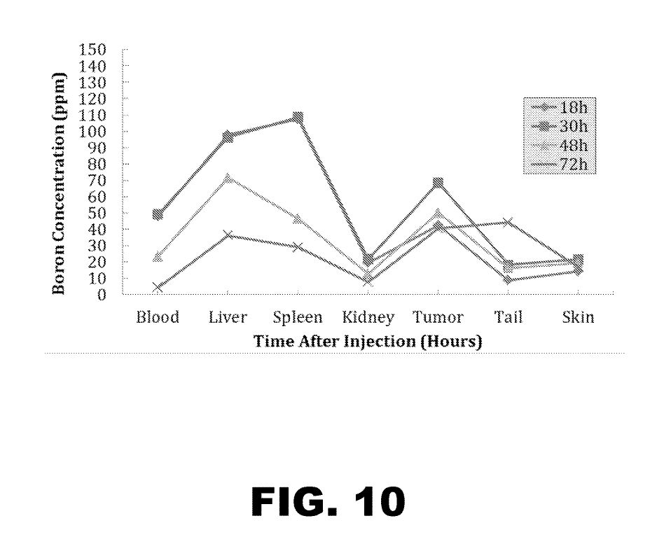

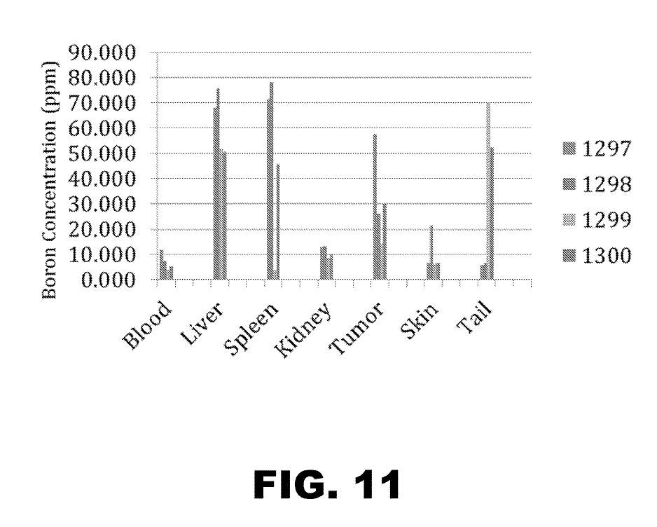

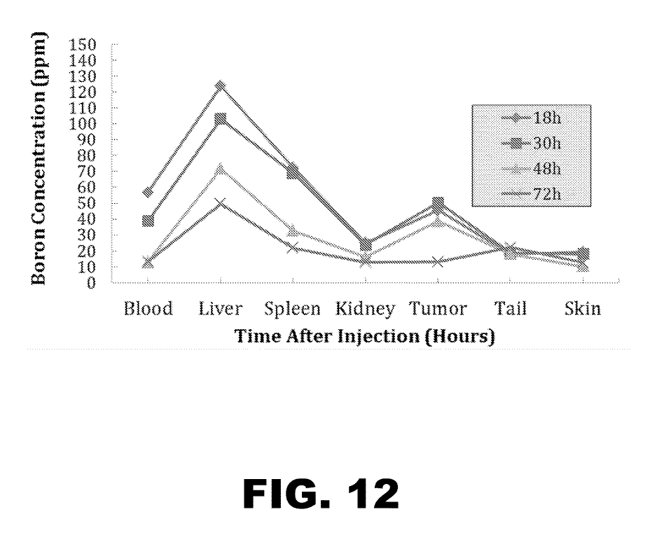

Comparison of boron biodistribution in tumor between 110 nm liposomes and 130 nm liposomes show some differences. FIGS. 10-12 show different biodistribution studies highlighting differences in the boron uptake in the tumor based on the liposme size. Specifically, TAC/MAC liposomes made by extrusion had a particle size of about 110 nm in monomodal particle distribution with a PDI of 0.05, and provided boron in the tumor at a concentration of about 60 ppm (FIG. 10). TAC/MAC liposomes with about 130-nm particles size made by extrusion had a PDI of 0.05, and provided boron in the tumor at a concentration of about 40 ppm (FIG. 11). TAC/MAC liposomes made by homogenization had a particle size of about 100 nm in bimodal particle distribution, and provided boron in the tumor at a concentration of about 60 ppm (FIG. 12).

Separation of the prepared liposomes from the solution was performed by a combination of tangential-flow ultrafiltration (TFF), where a membrane with very small pore size retained liposomes but allowed small solutes to pass through, and size exclusion (SE) gel filtration. An ultrafiltration cartridge with a 1 ft² polyethersulfone membrane with a molecular weight cutoff of 10 or 30 kD was typically used. In a typical protocol, a homogenized mixture of suspended liposomes and remaining free TAC solution was initially concentrated with TFF, followed by filtration through a size exclusion gel. The liposome containing fractions were concentrated again with TFF. The liposomes were then filtered with a Sterivex™ filter unit by filtering first through a 0.4-μm membrane and then a 0.20-μm membrane if possible.

Liposomes were analyzed by inductively coupled plasma-atomic emission spectroscopy (ICP-AES) for particle size, boron content, and TAC/MAC ratios. The particle size of the liposomes ranged from 100 nm to 140 nm as determined by dynamic light scattering at 25° C. Measurements using electrophoretic light scattering in water at 25° C. provided a ζ-potential of the liposome suspensions of −76.4±1.1 mV (n=4), indicating a high degree of resistance to aggregation. Liposomes encapsulating both TAC and MAC contained 1800 ppm boron and had a TAC:MAC molar ratio of 1:5.5 to 1:6. In addition, extrusion process yields a TAC:MAC molar ratio of 1:1.6 to 1:2.

### Example 3

### Cell Culture, Tumor Induction, and Experimental Design

EMT6 cells were purchased from American Type Culture Collection and cultured in Weymouth medium supplemented with 10% fetal bovine serum (FBS) as recommended by American Type Culture Collection. Cells in log phase were dissociated by incubation with TrypLE™ buffer (Life Technologies) for 10 min, followed by addition of FBS containing medium to terminate TrypLE digestion. Cells were then centrifuged at 323×g for 8 minutes at room temperature using Fisher Scientific Accuspin™ 3R centrifuge, and the cell pellets were resuspended in PBS solution. Cells were counted using a Countess Automatic Cell Counter (Life Technologies). For tumor production, EMT6 cells (1×106 cells/mouse) were inoculated into the right flank of female BALB/c mice having an average bodyweight of 20±1 g, following standard protocols. Animals were typically purchased in groups of 12 mice (Harlan Laboratories) for tumor inoculation and were assigned to the study groups described later contingent upon how quickly the tumors reached the target volume. All animal procedures were conducted according to protocols approved by the University of Missouri Animal Care and Use Committee.

### Example 4

### In Vitro Biodistribution of TAC/MAC Liposomes

In vitro biodistribution studies of the liposomes in EMT6 murine mammary carcinoma cell line show the localization of TAC in cytoplasm specifically in lysosomes and localization of MAC in the cell membrane. These biodistribution studies were done by incubating TAC/MAC liposomes with EMT6 cells for 24 h. After 24 h, the cells were washed to remove any excess of liposomes present in the culture media. The cells were then lysed and cell component separated by filtration. Cell membrane components and the cytoplasm components were separately analyzed for boron by ICP-OES and mass spectrometry methods. Mass spectrometric analysis showed that TAC remained in the cytoplasm and MAC remained in cell membrane.

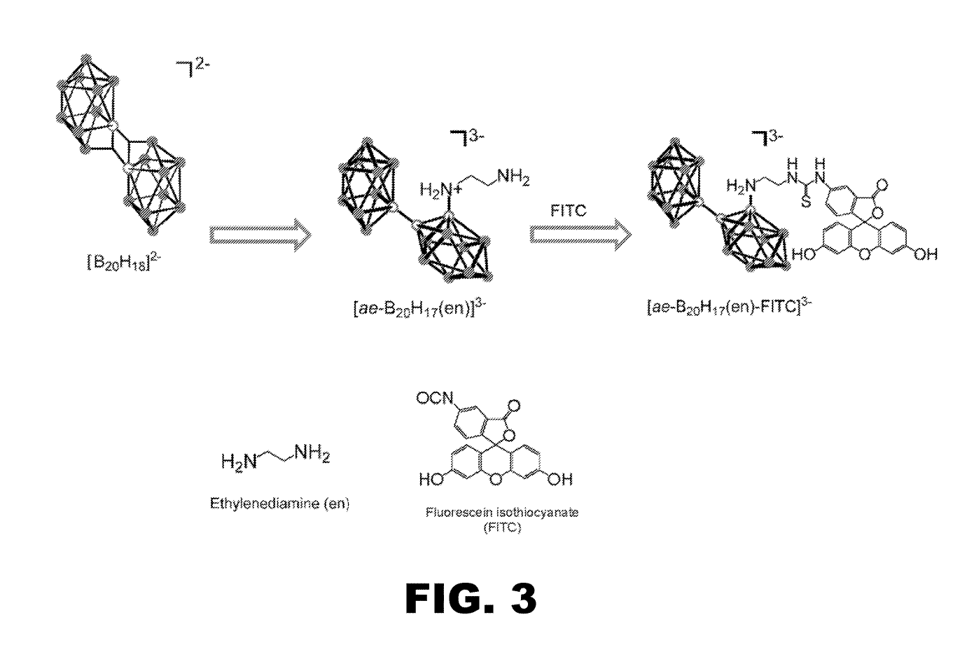

To investigate the location of TAC in the cell, a new fluorescent-labeled TAC analog was synthesized as shown in FIG. 3, and along with MAC was encapsulated in the liposome. The in vitro biodistribution of this liposomal formulation showed fluorescent-labeled TAC analog resides predominantly inside the lysosomes.

### Example 5

### In Vivo Biodistribution Studies

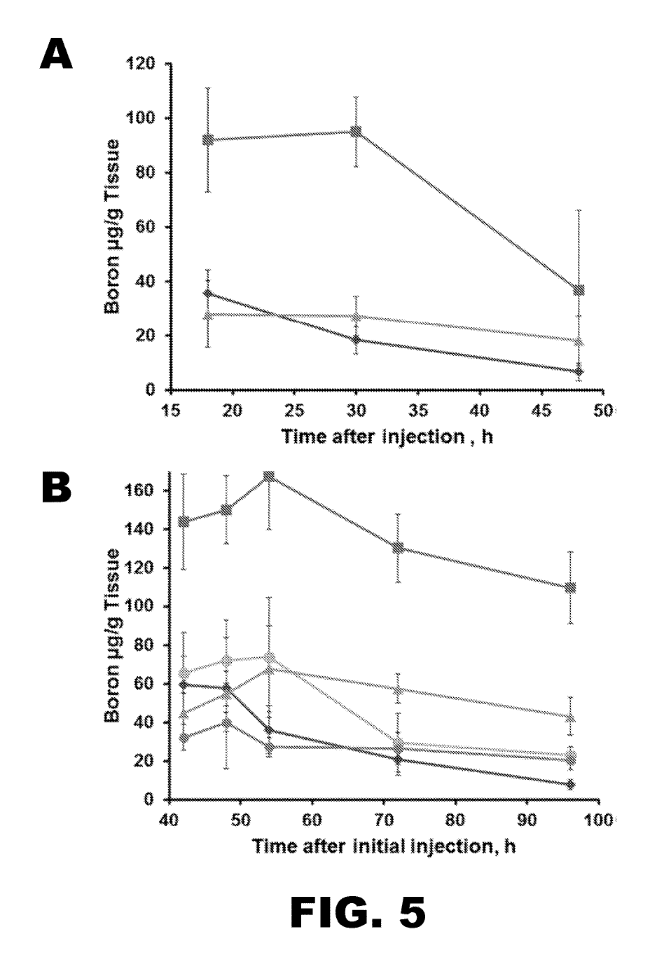

Experiments were performed to monitor the distribution of boron in tumor, blood, and normal tissues over time to identify the optimal injection protocol for delivery of boron to tumor tissue. FIG. 5A is a graph of the distribution of boron in mouse tissues over time following a single injection of TAC/MAC liposomal suspension (combination of two studies with injected doses of 345 μg or 340 μg of boron). FIG. 5B is a graph of the distribution of boron in mouse tissues over time following a double injection of TAC/MAC liposomal suspension (two single injections performed 24 hours apart totaling an injected dose of 742 μg of boron).

The first procedure consisted of a single 200-μL tail-vein injection of unilamellar liposomes incorporating TAC and MAC compounds [volume-weighted mean vesicle diameter (mv), 122 nm], with injected dose of boron being 342 μg (˜17 μg ¹⁰B per gram of body mass). Mice given a single injection were euthanized at 18, 30, or 48 hours after injection. Sufficient tissues were collected and analyzed to confirm previous biodistribution time-course results using this injection protocol in EMT6 tumor-bearing mice. The biodistribution results are shown in FIG. 5A. Tumor boron levels were sufficient for BNCT at 18 hours and 30 hours post injection (28.0 μg and 27.2 μg ¹⁰B per gram of tumor, respectively); however, the highest tumor/blood boron ratio (2.76:1) was not achieved until 48 h, when the boron concentration was only 18.1 μg ¹⁰B per gram tumor.

When the tumors had reached a target volume of 80 to 150 mm³, mice were administered boron-containing liposomes via lateral tail vein injection, and the distribution of boron in blood and organs was evaluated at specific intervals after injection. A double-injection protocol was tested in which two 200-μL injections of liposomes (mv of 106 nm; 371 μg of boron per injection, 18.6 μg ¹⁰B per gram body mass) were administered 24 hours apart. To examine the full biological impact of a double injection, boron content was assessed in multiple different tissues over a prolonged time interval. Mice given two injections were euthanized at 42, 48, 54, 72, or 96 hours following the initial injection. At each time point, brain, lung, heart, liver, kidney, spleen, tumor, blood, and tail samples were harvested and stored at −80° C. until they could be evaluated for boron content.

The resulting biodistribution of boron in murine tissues over time is shown in FIG. 5B. With the double injection protocol, higher peak boron concentrations were obtained in all tissues compared with a single injection because clearance from the first injection was not complete by 24 hours (FIG. 5A). Delayed clearance of boron from the tumors caused boron levels to peak in tumor tissue 54 hours after the first injection. Concentrations in liver and spleen also peaked at 54 hours; by contrast, boron levels in the blood were decreasing by 48 hours. At 54 hours, boron concentration in the tumors was 67.8 μg ¹⁰B per gram tumor, and the tumor/blood boron ratio was 1.88:1. As clearance of boron from blood proceeded more rapidly than loss from tumors, the tumor/blood ratio continued to increase after 54 hours. At 72 hours, the ratio was 2.76:1, and, by 96 hours, it had reached 5.66:1. The tumor boron concentrations at 72 hours and 96 hours were 57.4 μg ¹⁰B per gram tumor and 43.0 μg ¹⁰B per gram tumor, respectively.

A notable feature of the double-injection scheme is the wide window of time available for neutron irradiation—the concentration of boron in the tumors remains well within the therapeutic range for at least 96 hours after the initial injection while tumor/blood boron ratios continue to increase. Considering these results, the double-injection protocol was adopted for irradiation studies. In addition, the 54-h time point was chosen as most optimal for irradiation, with the initial assumption having been that a greater tumor boron concentration was the most influential factor in BNCT efficacy.

Tissues were digested by using a Microwave Accelerated Reaction System (Mars; CEM), and their boron content was determined via inductively coupled plasma optical emission spectroscopy (ICP-OES) with a PerkinElmer Optima™ 7000 DV in accordance with published methods. As a control to determine whether the tail vein injections were successful and therefore whether biodistribution data from a particular animal were valid, tails were routinely analyzed for boron content. Levels of boron in the blood were never observed to exceed 100 μg boron per gram blood; thus, if a tail value exceeded 100 μg boron per gram tail, the injection was assumed to have failed and that animal's data were excluded from the study.

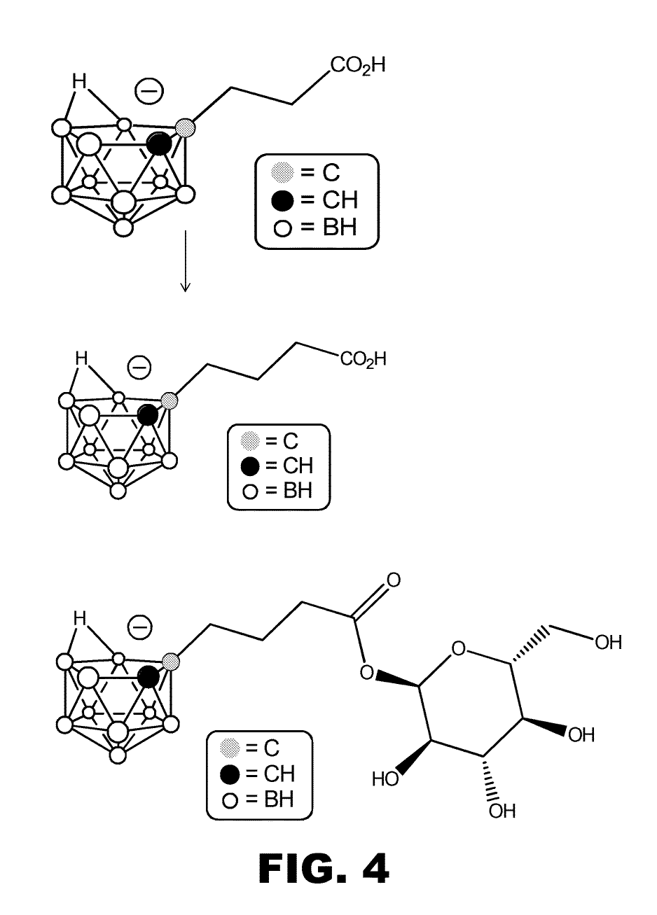

One of the components of in vivo biodistribution study of TAC/MAC liposomes in mice was identification of any metabolic transformation of either TAC or MAC in the body. Metabolic degradation of TAC was not observed in vivo. Similarly, a majority of MAC was expelled without change. Three main metabolites of MAC were identified in mouse urine by matrix-assisted laser desorption imaging mass spectrometry (MALDI-MS) analysis and are illustrated in FIG. 4.

### Example 6

### Therapeutic Effect Studies

When tumors had reached the 80-150 mm³ target volume, mice were administered ¹⁰B-enriched boronated liposome suspensions via tail-vein injections and subjected to thermal neutron irradiation following the protocol described in Example 7. The therapeutic effect of boron neutron capture therapy (BNCT) was evaluated based on changes in tumor volume over time. Neutron-only tumor-bearing mice were given no boron compounds but were exposed to the same irradiation protocol as the treatment group. Control mice were neither injected with a boron agent nor exposed to thermal neutron irradiation. Tumors on all mice were measured daily with calipers by the same observer throughout the course of an experiment. Measurements were taken until calculated tumor volume exceeded 2,000 mm³ or the longest diameter exceeded 20 mm. Mice were evaluated daily by a veterinarian or animal care staff for any evidence of ill thrift (failure to thrive) or restricted range of motion.

### Example 7

### Neutron Irradiation

Just before irradiation, mice were anesthetized by intraperitoneal (i.p.) administration of a combination of 10 mg/kg xylazine and 80 mg/kg ketamine. Cu/Au flux wires were set on the right and left thorax and flank of each mouse before it was placed in a positioning gantry. The gantry held a maximum of four mice or phantoms and permitted selective irradiation of the caudal or cranial half of an animal. To avoid any potential complications and improve the selectivity of the treatment, the head, thorax, and cranial abdomen of the mice were shielded by using ⁶LiCO₃ during irradiation. The gantry was placed in the irradiation chamber, and mice were irradiated with thermal neutrons for a maximum of 30 min. A camera in the irradiation chamber permitted observation of animals during treatment. Following irradiation, mice were removed from the gantry and allowed to recover from anesthesia. The Cu/Au flux wires were collected from the mice and counted by using a high-purity germanium γ-spectrometer, and saturation activities were assessed to confirm neutron flux to the animal.

The average flux of the neutron beam was determined to be 8.8×10⁸ neutrons/cm²·s (±7%) integrated over the thermal energy range of 0.0 to 0.414 eV. The measured cadmium ratio for gold was 130:1, indicating contamination with higher energy neutrons was minimal. Thus, for 30 minutes of the irradiation, the approximate background physical doses from hydrogen recoil and nitrogen capture interactions were 41.1 cGy and 34.2 cGy, respectively, and the approximate physical dose from boron capture was 12.9 cGy per 1 ppm of boron in tissue. The incident γ-component of the beam was estimated to be about 63.6 cGy, with a small additional γ-component induced by thermal neutron capture in hydrogen. Therefore, assuming a tumor boron concentration of 60 ppm, the physical boron neutron capture dose was about 7.75 Gy, well greater than the background components from all sources.

Dose-escalation studies were conducted by varying the duration of neutron irradiation. Three data sets were collected, including a single irradiation study of 30 minutes duration, a double irradiation study whereby a 30-min irradiation was repeated after 7 days (following another double injection), and a single irradiation of 60 minutes duration. In the 30-min single-irradiation study, liposomes (mv, 130 nm) containing ¹⁰B-enriched TAC and MAC were injected into the tail vein of 12 treatment mice following the double-injection protocol at a dose of 342 μg boron (17.1 μg ¹⁰B per gram body mass) per injection. At 54 hours after the initial injection, the mice were irradiated for 30 min, resulting in a total fluence of 1.6×10¹² neutrons/cm². Fourteen “neutron only” mice that received no injections of any kind were irradiated under the same conditions. Both groups of mice were compared with an untreated control group of 23 mice that received neither injections nor irradiation.

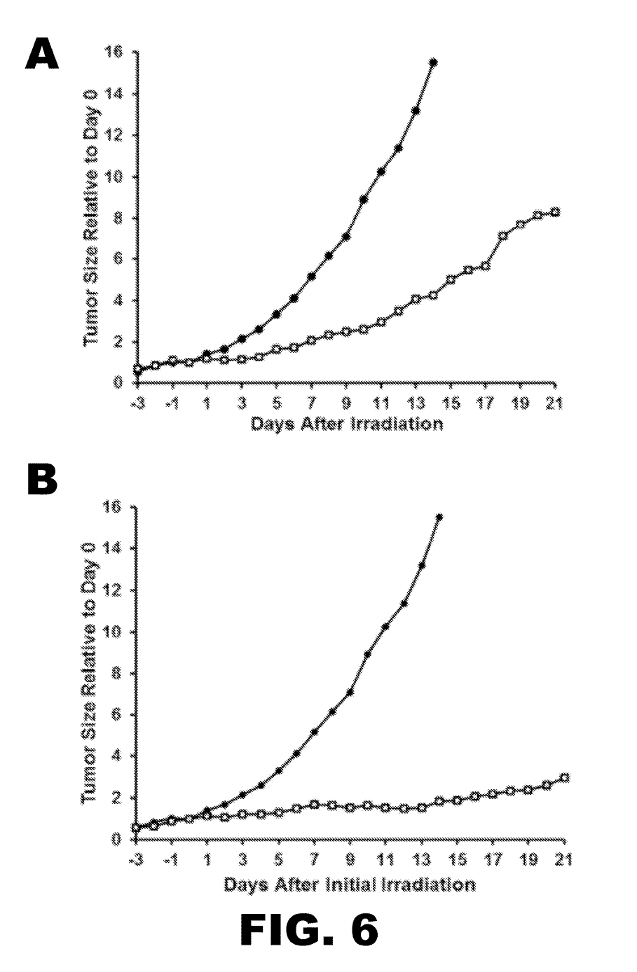

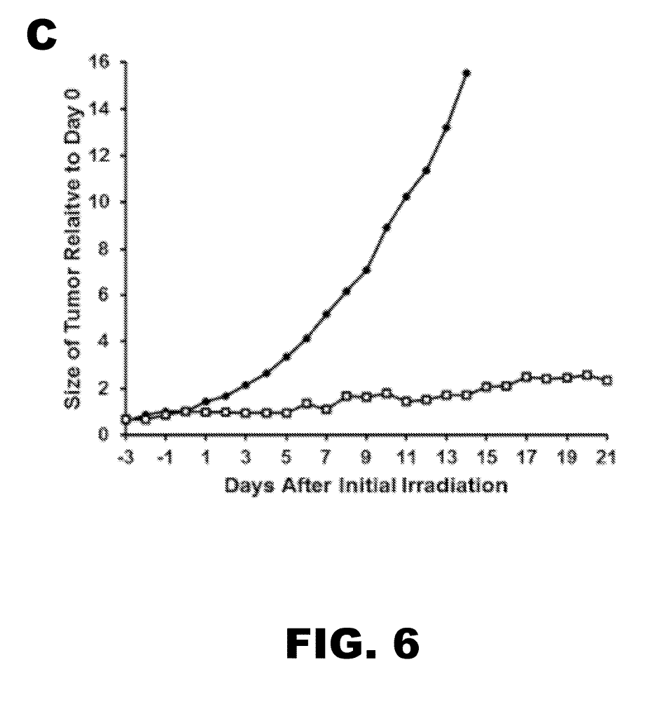

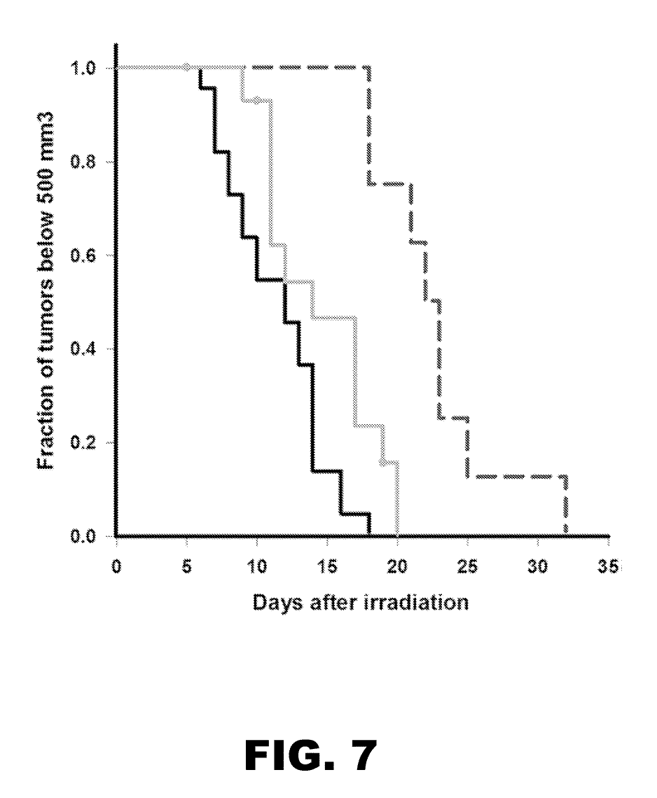

FIG. 6A is a graph of tumor growth normalized with respect to average volume at day 0 for a single BNCT treatment consisting of a 30-min irradiation following double injection of liposomal suspension. FIG. 6A demonstrates that, in mice given a single 30-min BNCT treatment, tumor growth was significantly slower compared with untreated controls. The rapid rate of tumor enlargement and concomitant decline in the health status of the non-treated control mice prevented assessments of tumor volume beyond 14 days post irradiation. By 14 days, tumor volume in the control mice had increased by 1,551%, whereas the volume in mice given BNCT had increased by only 424%. The tumor volume increase in neutron-only mice was 737%, suggesting some inhibitory effect from irradiation alone. Therefore, further evaluation by Kaplan-Meier time-to-event analysis was conducted (FIG. 7). The median time for tumors to reach a volume of 500 mm³ in mice given BNCT was 22 days, statistically significant at the 5% level compared with 14 days for mice receiving neutron irradiation only (P<0.002) or 12 days for the untreated control mice (P<0.001). Comparison of the median tumor growth times of neutron-only mice with untreated controls was significant at the 5% level but not at the 1% level (P=0.012).

In the next study, the effect of two identical rounds of BNCT was examined in an experiment in which four mice were administered a second treatment 7 days after the first. Mice were given another double injection by using the same liposome suspension described earlier, and the mice were again irradiated for 30 minutes at 54 hours after the initial injection. FIG. 6B is a graph of the tumor growth normalized with respect to average volume at day 0 for two single BNCT treatments (double injection of liposomal suspension, 30 minutes irradiation) performed 7 days apart. FIG. 6B demonstrates that retreatment had an additional impact on tumor growth. At 14 days post irradiation, tumor volume had increased only by 186%; even by 21 days, volume increase was only 297%.

To determine whether neutrons were a limiting factor, the effect of a longer duration of neutron irradiation on tumor growth was examined. Four mice were given a double injection of liposomes (mv, 113 nm; injected dose, 351 μg of boron per injection; 17.6 μg ¹⁰B per gram body mass) and, at 54 hours after the initial injection, mice were irradiated for 30 minutes Immediately following the irradiation, mice were allowed to recover from anesthesia for 15 minutes before being anesthetized again and irradiated for another 30 minutes. This procedure effectively doubled the amount of time the mice were irradiated at the 54-h time point of the double-injection treatment protocol. FIG. 6C is a graph of tumor growth normalized with respect to average volume at day 0 for a single BNCT treatment consisting of a 1-h irradiation following double-injection of liposomal suspension. Although the data set was limited as a result of excessive difficulty in recovering animals from the second round of anesthesia, the results show that doubling the irradiation time leads to additional suppression of tumor growth (FIG. 6C). Tumor volume increase at 14 day was 169%, and at 21 day was 233%, a reduction comparable to that obtained when two rounds of BNCT were administered 1 week apart (FIG. 6B).

None of the mice exhibited any apparent deleterious effects from BNCT. No toxic effects from liposomal injections were detected, and no radiation side effects were observed, even in those mice exposed to the neutron beam for 1 h. Although the inability to identify any effects on the skin or in other organs precluded calculation of a relative biological effectiveness for the neutron beam or a compound biological effectiveness for the boronated liposomes, the lack of observable detrimental effects is encouraging because it allows for the possibility of further boron and neutron dose escalation. Dose-limiting side effects have been encountered in several human and animal trials of BNCT that could at least in part be attributed to the relatively high relative biological effectiveness of the background epithermal, fast neutron, and γ-spectral components of the neutron beams. The thermal neutron beam constructed for these BNCT examples possesses minimal γ- and fast neutron contamination.

These experiments establish suppression of tumor growth by BNCT following delivery of therapeutic quantities of boron to tumors via liposomes carrying polyhedral boranes and carboranes. Alternative systems are being investigated in which the therapeutic agent is covalently combined with the delivery system to increase boron uptake, enhance stability, and even circumvent the need for ¹⁰B-enrichment of the boron compounds. Nanoparticles with these features are presently being designed and will offer the capability of facile surface modification to increase circulation time, apply a specific targeting mechanism, allow for additional ease of scalability, and reduce cost of therapy.

### Example 8

### Data Analysis

Tumor volumes were calculated by using the equation

$$V = \frac{π}{6} * 6 * I * w * d ,$$

where l is the length (greatest longitudinal diameter), w is the width (greatest transverse diameter), and d is the diameter (greatest diameter orthogonal to the plane formed by l and w) for an elliptical tumor in millimeters. Time-to-event curves were estimated by using the Kaplan-Meier method, and outcomes among treatment groups were compared using the log-rank test. Biodistribution plots and growth curves were constructed in Microsoft Excel 2007, and survival analysis was conducted by using SigmaPlot 12.0.

While specific embodiments have been described above with reference to the disclosed embodiments and examples, such embodiments are only illustrative and do not limit the scope of the disclosure. Changes and modifications can be made in accordance with ordinary skill in the art without departing from the disclosure in its broader aspects as defined in the following claims.

What is claimed is:

1. A composition for boron neutron capture therapy, comprising a liposome having a mean diameter ranging from 100 nanometers to 140 nanometers, the liposome having an aqueous core and an encapsulating bilayer, wherein the aqueous core comprises Na₃[1-(2′-B₁₀H₉)-2-NH₃B₁₀H₈] (TAC) and the encapsulating bilayer comprises K[nido-7-CH₃(CH₂)15-7,8-C₂B₉H₁₁] (MAC); and wherein total molar ratio of TAC to MAC in the liposome is from 1:1 to 1:6.

2. The composition of claim 1, wherein the boron content per liposome is from 1600 ppm to 1900 ppm.

3-4. (canceled)

5. The composition of claim 1, wherein the outer charge of the liposome has a zeta potential ranging from −70 mV to −80 mV.

6. (canceled)

7. The composition of claim 1, wherein the liposome comprises cholesterol and 1,2-distearoyl-sn-glycero-3-phosphocholine.

8. The composition of claim 1, wherein total molar ratio of TAC to MAC in the liposome is from 1:3.6 to 1:6.

9. The composition of claim 1, wherein total molar ratio of TAC to MAC in the liposome is from 1:5.5 to 1:6.

10. The composition of claim 1, wherein at least one of TAC and MAC is enriched with ¹⁰B.

11. A method of inhibiting growth of a tumor in a subject in need thereof, the method comprising:
a) administering to a subject in need thereof a liposome having a size ranging from 100 nanometers to 140 nanometers, the liposome having an aqueous core and an encapsulating bilayer,
wherein the aqueous core comprises Na₃[1-(2′-B₁₀H₉)-2-NH₃B₁₀H₈] (TAC) and the encapsulating bilayer comprises K[nido-7-CH₃(CH₂)15-7,8-C₂B₉H₁₁] (MAC); and
wherein total molar ratio of TAC to MAC in the liposome is 1:1 to 1:6; and
b) irradiating the tumor in the subject with neutrons.

12. The method of claim 11, wherein the time between initial administration of the liposome and irradiation of the tumor is greater than 56 hours.

13. The method of claim 11, wherein the time between initial administration of the liposome and irradiation of the tumor is greater than 96 hours.

14. The method of claim 11, wherein the ratio of boron in the tumor to the boron in the blood reaches greater than 5:1 prior to irradiation of the tumor.

15-16. (canceled)

17. The method of claim 11, wherein the tumor has an increase in volume of at least 500% less than the increase in volume of a similar tumor in a control, untreated subject after 14 days.

18. The method of claim 11, wherein total molar ratio of TAC to MAC in the liposome is from 1:3.6 to 1:6.

19. The method of claim 11, wherein total molar ratio of TAC to MAC in the liposome is from 1:5.5 to 1:6.

20. The method of claim 11, wherein at least one of TAC and MAC is enriched with ¹⁰B.

21. A method for preparing K[nido-7-CH₃(CH₂)ₙ-7,8-C₂B₉H₁₁], the method comprising
a) contacting a compound comprising [B₁₀H₁₀]²⁻ with a strong acid and dialkylsulfide to give a sulfur-containing B₁₀H₁₂ intermediate;
b) contacting the sulfur-containing B₁₀H₁₂ intermediate with an alkyl alkyne to form a carborane; and
c) contacting the carborane with a strong base to give K[nido-7-CH₃(CH₂)ₙ-7,8-C₂B₉H₁₁].

22. The method of claim 21, wherein the strong acid is CF₃SO₃H.

23. The method of claim 21, wherein the dialkylsulfide is diethylsulfide.

24. The method of claim 21, wherein the sulfur-containing B₁₀H₁₂ intermediate is B₁₀H₁₂(Et₂S)₂.

25. The method of claim 21, wherein the alkyl alkyne is CH₃(CH₂)₁₅CCH.

26-31. (canceled)
